(19) United States
(12) Patent Application Publication
(10) Pub. No.: US 2025/0026700 A1
(43) Pub. Date: Jan. 23, 2025

(54) Combined Capture and Conversion of CO2 to Methanol in a Post Combustion Capture Solvent

(71) Applicant: BATTELLE MEMORIAL INSTITUTE, Richland, WA (US)

(72) Inventors: Jotheeswari Kothandaraman, Richland, WA (US); Johnny Saavedra Lopez, Richland, WA (US); Yuan Jiang, Richland, WA (US); Robert A. Dagle, Richland, WA (US); David J. Heldebrant, Richland, WA (US)

(21) Appl. No.: 18/354,571

(22) Filed: Jul. 18, 2023

(51) Int. Cl. C07C 29/153; B01D 53/14; B01D 53/62; B01D 53/78; B01J 21/06; B01J 23/10; B01J 23/42; B01J 23/63; C01B 32/40

(52) U.S. Cl. CPC C07C 29/153; B01D 53/1475; B01D 53/1493; B01D 53/62; B01D 53/78; B01J 21/063; B01J 23/10; B01J 23/42; B01J 23/63; C01B 32/40; B01D 2252/202; B01D 2252/2041; B01D 2252/20426; B01D 2252/20431; B01D 2252/20452; B01D 2252/504; B01D 2257/504

(57) ABSTRACT

An efficient and selective catalyst for the condensed phase hydrogenation of captured CO₂ in the presence of an advanced water-lean post combustion capture solvent, EEMPA. Selected heterogenous catalysts for suppress N-methylation of the capture solvent; especially a noble metal on a reducible metal oxide support is highly selective for C—N cleavage to produce methanol.

### STATEMENT REGARDING FEDERALLY-SPONSORED RESEARCH AND DEVELOPMENT

This invention was made with Government support under Contract DE-AC0576RL01830 awarded by the U.S. Department of Energy. The Government has certain rights in the invention.

### Introduction

Continuous anthropogenic emissions of CO₂ into the atmosphere since the industrial revolution have been associated with the ongoing environmental issues such as global warming and climate change. While we transition from fossil energy to renewable energy sources to reduce the CO₂ emissions, massive deployment of carbon capture, utilization, and storage (CCUS) technologies are needed to limit the global temperature rise (1.5-2° C.). Thus, direct air capture and flue gas capture technologies have been gaining considerable attention lately. Compared to direct air capture (containing ˜400 ppm CO₂), post-combustion CO₂ capture from industrial flue gas (containing 5-15% CO₂) is an efficient and economic approach to curtail the emissions to the atmosphere from industrial sources, such as fossil-powered power plants, cement, and steel industries. Particularly, the solvent based post-combustion CO₂ capture technologies using amines (e.g., monoethanolamine) have been adopted commercially.

Upon the release of the captured CO₂ via thermal regeneration of the capture solvent, the CO₂ can either be stored underground for permanent CO₂ storage or it can be used as a C₁ source to produce value-added chemicals/materials and fuels, such as formic acid, methanol, methane, ethanol, polycarbonates, and others. However, the overall process of capture, compression, transportation, and storage is not energy efficient and cost effective. To overcome these challenges, the integrated capture and conversion of captured CO₂ to materials (IC³M) has been developed. In the conventional CCUS approach, the cost of the capture process is mostly dominated by the energy intensive regeneration of the capture solvent. In the IC³M approach, the exothermic conversion process partially offsets the energy required for the regeneration of the capture solvent. In addition, because the captured CO₂ is converted directly in the capture solvent medium, there is also energy saved on the CO₂ compression and transportation, thus reducing additional energy inputs, and associated equipment cost. In the IC³M process, CO₂ is directly converted to methanol on the IC³M solvent avoiding energy-intensive mechanical compression of the CO₂. By reducing the need for these additional energy inputs, and the associated equipment, IC³M becomes a viable technology for modular distributed-scale processing platforms, which in turn could enable distributed applications such as the separation and conversion of CO₂ from landfill gases, waste-water treatment gases, and manure off-gas. Other sources of CO₂ may include, but are not limited to, fermenters, cement plants, steel kilns, and pulp and paper mills. Stranded hydrogen sources are also more likely to be co-sourced when considering distributed processing, which could enable lower cost/renewable hydrogen supplies in many applications.

Among the CO₂ derived value-added chemicals, methanol is most attractive as a fuel and a chemical feedstock to produce olefins, aromatics, and other chemicals. Currently, methanol is produced industrially from CO₂/CO and H₂ mixture using a ternary copper-based catalyst (Cu/ZnO/Al₂O₃) catalyst in the gas phase at high reaction temperature (>200° C.) and pressure (70-100 bar). In traditional gas phase catalysis, there are three proposed mechanisms involving (a) formate (HCOO*) intermediate, (b) CO* intermediate via reverse water gas shift mechanism (RWGS), and (c) trans-hydrocarboxyl (COOH). These intermediates are suggested depending on the catalysts and conditions (FIG. 1. (A), formate and CO pathways).^{[1]} In the condensed phase hydrogenation in the presence of amine-based capture solvent, the intermediates involved are ammonium carbamate, formate and N-formamide (FIG. 1, (B)). The final hydrogenation of the N-formamide intermediate can form many products such as methane, methanol and N-methylation of the capture solvent. The N-methylation of the capture solvent causes solvent deactivation and thereby the active —NH— unit of the capture solvent is unavailable for subsequent reuse for CO₂ capture. The C—N cleavage of the N-formamide intermediate form methane or methanol, while the C—O cleavage cause N-methylation.

Despite these and other efforts, there remains a need in the industry to develop cost-effective, environmentally friendly CO₂ conversion catalyst systems that are commercially viable.

### SUMMARY OF THE INVENTION

C—N cleavage of the N-formamide intermediate forms methane or methanol, while C—O cleavage causes N-methylation. See FIG. 1. Upon evaluating various metal with different supports, we identified that metals, particularly noble metals on reducible metal oxide supports such as CeO₂ and TiO₂ to be particularly selective for C—N cleavage to produce methanol, which was supported by operando ¹³C NMR studies.

We have demonstrated an integrated capture and utilization approach to produce methanol directly from the captured CO₂ inside an economically viable water-lean post-combustion capture solvent, that in time, may achieve cost parity with fossil-derived methanol. We have identified that traditional gas-phase CO₂ hydrogenation catalysts deactivated the solvent via N-methylation of the 2° amine moiety. We discovered that the Pt catalysts on reducible metal oxides (such as TiO₂ or CeO₂) suppress undesirable N-methylation of the capture solvent, showing high selectivity for C—N bond cleavage. The most promising catalyst, Pt/TiO₂, with its known favorable chemical stability in organic solvents, strong metal-support interaction, and acid-base properties were further evaluated under continuous-flow operation, which produced a single-pass CO₂ conversion of 29% with 70% selectivity to methanol at 170° C. Further increase in the reaction temperature to 190° C. resulted in a decrease in methanol selectivity with an unprecedented single-pass CO₂ conversion of 86%. The high single pass conversion achieved here is important as the solvent looping strategy to get higher conversion is not economical. Technoeconomic analyses (TEA) performed on the current state of this integrated technology showed that the methanol production cost is ˜$1000/mt using CO₂ captured from flue gas from a 550 MW natural gas combined cycle (NGCC) plant. This cost is lower than the current renewable methanol cost of ˜$1600/mt. Selectivity % refers to mol % throughout all the descriptions.

In one aspect, the invention provides a method of converting CO₂ to methanol comprising: contacting a solution comprising CO₂ in an amine sorbent over a solid catalyst comprising a noble metal on a reducible metal oxide support at a temperature in the range of 181° C. to 225° C.; wherein the CO₂ is post or pre combustion CO₂; and wherein the conversion of CO₂ occurs in a condensed phase and the CO₂ is converted to methanol with at least 40% selectivity; wherein the single pass conversion of CO₂ is at least 75%; and wherein the C₂₊ alcohol selectivity, or the ethanol selectivity, is at least 4 mol %.

In another aspect, the invention provides a method of converting CO₂ comprising: providing continuous flow reactor system comprising: an absorber; a pump; and a reactor; enriching an amine sorbent with CO₂ by contacting the solvent with a flue gas comprising CO₂ to create a CO₂-enriched solution in the absorber; pressurizing the CO₂-enriched solution to between 30 and 100 bar H₂; heating the CO₂-enriched solution; producing a heated and pressurized CO₂-enriched solution; feeding the heated and pressurized CO₂-enriched solution with pressurized H₂ gas into a reactor comprising a solid catalyst comprising a noble metal on a reducible metal oxide support and reacting the solution over the solid catalyst at a temperature of between 181 and 225° C.; and producing a product solution in which the CO₂ in the solution is converted into methanol, C₂₊ alcohols, CH₄, and light hydrocarbons over the solid catalyst.

In any of its aspects, the invention can be characterized by one or any combination of the following: further comprising one or any combination of the following: wherein the methanol selectivity is at least 50%; wherein the methanol selectivity is in the range of 40 to about 80%; wherein the single pass conversion of CO₂ is in the range of 75 to about 90%; wherein the selectivity to ethanol is 4 to 10 mol %; wherein the selectivity to butanol is at least 1.7 mol %; wherein the selectivity to butanol is about 2 to about 5 mol %; wherein the conversion of CO₂ is in a continuous flow operation; wherein the method occurs in a single pass; comprising a single pass CO₂ conversion of 80-90%; wherein the catalyst support comprises CeO₂ or TiO_{2.}; wherein the amine sorbent is a water-lean solvent; further comprising separation and conversion of CO₂ from landfill gases, waste-water treatment gases, manure off-gas, fermenters, cement plants, steel kilns, and pulp and paper mills; wherein the amine sorbent is N-(2-EthoxyEthyl)-3-MorpholinoPropan-1-Amine (2-EEMPA); wherein the amine sorbent with 0.5-20 wt % CO₂ loading; wherein ethanol is a co-solvent; wherein CO and N-formamide are formed as intermediates; wherein the noble metal is Pt; wherein the solid catalyst comprises 0.1-20 wt % Pt, preferably 5-10 wt % Pt, on a CeO₂ or TiO₂ support; comprising a gas feed of 0.005-0.05 mL/min of H₂/N₂ gas; wherein the solid catalyst was pretreated in situ at 120° C. under a reducing flow of 30-65 sccm 10% H₂ in N₂; wherein the C—N cleavage selectivity is at least 50%, or preferably at least 80%, or between 85% and 100%; wherein the temperature is at least 185° C. or at least 190° C., or in the range of 185 to 195° C.; comprising a single pass CO₂ conversion of 80-90%; wherein the conversion of CO₂ occurs in a condensed phase; wherein the solid catalyst comprises Pt disposed on a reducible metal oxide support; wherein the catalyst supports comprise CeO₂ and/or TiO₂; wherein the amine sorbent is a water-lean post-combustion solvent; comprising the separation and conversion of CO₂ from landfill gases, waste-water treatment gases, manure off-gas, fermenters, cement plants, steel kilns, and pulp and paper mills; wherein the amine sorbent is N-(2-EthoxyEthyl)-3-MorpholinoPropan-1-Amine (2-EEMPA); wherein the amine sorbent is a solvent with 2-15 wt % CO₂ loading; wherein ethanol is a co-solvent; wherein CO and N-formamide are formed as intermediates; wherein the noble metal is Pt; wherein the solid catalyst comprises 5-10 wt % Pt disposed on a CeO₂ or TiO₂ support; wherein the gas flow is between 0.005-0.05 mL/min; wherein the catalyst is 27-100% selective for C—N bond cleavage; wherein the H₂ that was not consumed in the reactor is separated from the product solution in a pressure swing adsorption unit and recycled back to the reactor; wherein the methane and other light hydrocarbons are separated from the product solution in a pressure swing adsorption unit and used as fuel gas; wherein the alcohols are separated and recovered from the product solution;

wherein the alcohols and water are separated from the product solution and one another using extractive distillation, wherein ethylene glycol is used as an entrainer; wherein the CO₂-enriched solution contains 2-15% CO₂; and/or wherein the temperature is at least 185° C. or at least 190° C.

In another aspect, the invention provides a composition of matter comprising an amine solvent, CO₂, and a heterogenous catalyst, and methanol derived from the carbon dioxide, wherein the methanol derived from the CO₂ makes up at least 1% of mass percent of the CO₂ and wherein the molar ratio of the methanol produced from CO₂ to ethyl formate in the composition is greater than 2.

The invention can be further characterized by one or any combination of the following features: wherein the catalyst suppresses N-methylation of capture solvent; the composition exists in the condensed phase; wherein the solvent is a water-lean post combustion solvent; wherein the solid catalyst comprises a noble metal disposed on a reducible catalyst support.

The invention also includes a system comprising any of the compositions and conditions described herein. The invention can be further elucidated in the examples below. The invention may be further characterized by any features in the examples, for example, any of the inventive aspects can be further characterized by values within ±20% (or within ±10%) of any of the values in any of the examples, tables or figures; however, the scope of the present invention, in its broader aspects, is not intended to be limited by these examples.

The invention is often characterized by the term “comprising” which means “including,” and does not exclude additional components. In narrower aspects, the term “comprising” may be replaced by the more restrictive terms “consisting essentially of” or “consisting of.”

### Glossary

Absorber—A chamber adapted to provide space for contacting the amine solvent with the CO₂-containing gas.

Aliphatic—A substantially hydrocarbon-based compound, or a radical thereof (e.g., C₆H₁₃, for a hexane radical), including alkanes, alkenes, alkynes, including cyclic versions thereof (cycloaliphatic), and further including straight and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted.

Alkoxyalkyl—A radical having the general formula —RO—R′ where R and R′ independently are alkyl.

Amine—A compound including an amino group —N(R)R′ where R and R′are independently hydrogen, alkyl, heteroalkyl, haloalkyl, aliphatic, heteroaliphatic, aryl (such as optionally substituted phenyl or benzyl), heteroaryl, alkylsulfano, or other functionality. A 1°, or primary, amine includes an —NH₂ group. A 2°, or secondary, amine includes an —N(H)R group. A 3°, or tertiary, amine includes an —N(R)R′ group. A polyamine is an organic compound having more than two amino groups.

Amine Sorbent—A solvent containing an amine that is capable of retaining and releasing CO₂. The amine may comprise a 1° amine group, a 2° amine group, a 3° amine group, a pyridine, or any combination thereof. In any of the foregoing or following embodiments, the CO₂ may be adsorbed, absorbed, covalently, or ionically bound to the amine. In one embodiment, the sorbent comprises a 2° amine and a pyridine on the same molecule. In some embodiments, the amine comprises a polyamine, a tertiary amine, a compound according to Formula I, or any combination thereof, the compound according to Formula I having a structure R¹(R²)N-L¹-NH—R³ where each of R¹ and R² independently is aliphatic or cycloaliphatic or R¹ and R² together with the nitrogen to which they are attached form a heterocyclic ring, L¹ is aliphatic or cycloaliphatic or L¹ and R¹ together with the nitrogen to which they are attached form a heterocyclic ring, and R³ is aliphatic, cycloaliphatic, cycloalkylalkyl or alkoxyalkyl.

Aromatic or aryl—A monovalent aromatic carbocyclic group of, unless specified otherwise, from 6 to 15 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

C—N bond cleavage selectivity—This measure is calculated using the following: C—N cleavage selectivity=(moles of methanol)×100/(moles of EEMPA-N-CH₃+moles of methanol). In the general case, EEMPA-N-CH₃ can be replaced by the sorbent.

CO₂-enriched—This is a relative term indicating that the substance has a higher concentration of CO₂ than a previous state. In preferred embodiments, a CO₂-enriched substance comprises at least 2 wt % CO₂ or at least 10 wt % CO₂, or in the range of 5-15 wt % CO₂.

CO₂-lean—This is a relative term indicating that the substance has a lower concentration of CO₂than the CO₂-enriched state. In some embodiments, less than 2 wt % CO₂, or in some embodiments 1 wt % or less.

Condensed phase—The term “condensed phase” refers to a liquid or solid phase, including liquid/liquid, liquid/solid, and solid/solid solutions and mixtures.

Heterocycle/Heterocyclic—A heterocycle or heterocyclic molecule comprises a 5-6 member ring wherein 4-5 member atoms are carbon atoms and 1-2 member atoms are heteroatoms, which may be sulfur, nitrogen, or oxygen, or any combination thereof.

Methanol selectivity—The mole % of converted CO₂ that was been converted into methanol. For example, if 2 moles of CO₂ have been consumed and 0.5 moles of methanol have been produced, then the selectivity is 25%.

Noble metal—Preferably, noble metals include gold, platinum, ruthenium, rhodium, palladium, osmium, iridium, and any combination thereof, but may also include copper and silver, and any combination of any of the above.

Reducible metal oxide support—The term “reducible oxides” is generally known to chemists interested in heterogeneous catalysis. Reducible oxides are solid state materials that are strongly affected by the reversible oxidation state of the metal. Transition metal oxides such as CeO₂ and TiO₂ are considered reducible oxides because the conditions required for thermodynamically favorable reducibility are attainable. On the other hand, for oxides such as Al₂O₃ and MgO, these conditions are quite extreme and therefore these catalyst supports are considered to be non-reducible. Reducible oxides include, but are not limited to CeO₂, TiO₂, VₓO_{y}, FeₓO_{y}, CoₓO_{y}, HfO₂, ZrO₂, MnOₓ, PrOₓ, SmOₓ, MoO₃, WO₃, In₂O₃, and more complex solids, such as BaTiO₃ and LaCoO₃, and any combination thereof.

Water-lean—Any water content below 1 mole of water per mole amine, or below 1 molar equivalent, or below 10 wt %, preferably 1-5 wt %. Water content is commonly below 10 wt %, but most water-lean solvents operated for the carbon capture at 5 wt % or below water content. Compared to the aqueous post-combustion solvents (e.g., aqueous 30 wt % monoethanol amine), water-lean post-combustion solvents are superior for the IC3M approach mostly due to the following advantages: (1) high CO₂ physical solubility in the water-lean organic solvent, thus requiring low CO₂ pressure and temperature for the conversion. Lowering the reaction temperature can also increase the conversion and selectivity in hydrogenation reactions because it can suppress the endothermic reverse water gas shift reaction (ΔH=41.2 KJ/mol), (2) In the case of CO₂ hydrogenation (to methane, methanol, ethanol, methyl formate and others) reactions involving water as a byproduct, the excess water in the aqueous solvents could reverse the equilibrium and could also block catalyst active sites, and (3) water-lean solvents are more resistant to corrosion and solvent decomposition.

### BRIEF DESCRIPTION OF THE DRAWINGS

FIG. 1: Proposed gas phase (A) and condensed-phase (B) methanol synthesis from CO₂ and H₂.

FIG. 2: Reaction mechanism of post-combustion solvent system.

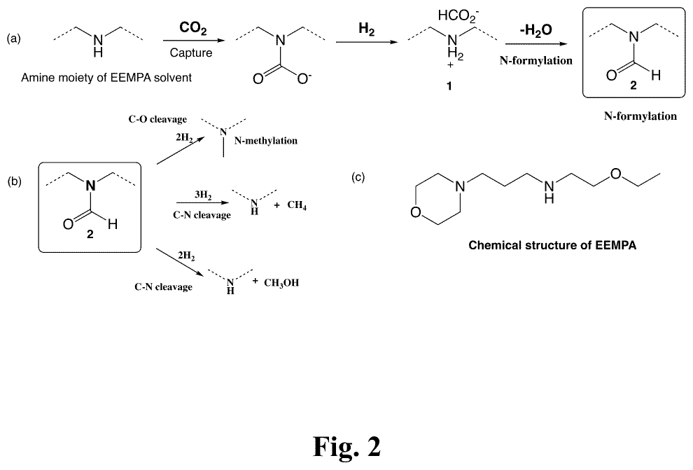

FIG. 3: Process flow diagram of the IC³M CO₂ capture and conversion to methanol process.

FIG. 4: Hydrogenation of captured CO₂ in the presence of Cu- and Ag-supported heterogeneous catalysts using a batch reactor system.

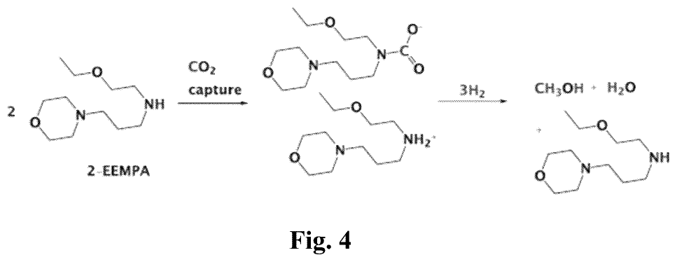

FIG. 5: Reaction mechanism with ethanol co-feed.

FIG. 6: In situ ¹³C MAS-NMR during the hydrogenation of ¹³CO₂ in the presence of 2-EEMPA and the Cu/ZnO/Al₂O₃ catalyst at 170° C. under 60 bar H₂ (initial pressure) in ethanol co-solvent, 2-EEMPA:EtOH=1:10 (molar ratio).

FIG. 7: NH₃-TPD (FIG. 7A) and CO₂-TPD (FIG. 7B). Conditions: Ammonia (10 mol % NH₃ in He) and CO₂ (10 mol % CO₂ in He) were adsorbed on 100 mg of sample. We loaded 100 mg of sample and purged under flowing He (25 sccm) for 10 min. Samples were pretreated in situ at 200° C. with 10% O₂/He for 2 h, followed by reduction at 200° C. with 10% H₂/N₂ for 20 min, He purge at 200° C. for 10 min, then cooled to 40° C. for NH3 or CO2 chemisorption. After the adsorption, samples were purged under flowing He (25 sccm) for 10 min, then the samples were heated up to 500° C. at a rate of 10° C./min. Resulting NH₃ and CO₂ uptakes are presented in Table 4. (TPD=temperature programmed desorption)

FIG. 8: ¹H NMR of the crude reaction mixture (from Table 5, entry 8) in CD₃CN.

FIG. 9: Proposed reaction mechanism for the hydrogenation of captured CO₂ in the presence of Pt/TiO₂ catalyst. Surface hydroxyl groups are not shown for simplicity.

FIG. 10: In situ ¹³C MAS-NMR during the hydrogenation of ¹³CO₂ in the presence of 2-EEMPA and a 5wt % Pt/TiO₂ catalyst at 170° C. under 60 bar H₂ (initial pressure) in an ethanol co-solvent, 2-EEMPA:EtOH=1:10 (molar ratio).

FIG. 11: XRD spectra of fresh and spent Pt/TiO₂ catalysts used in experiments in Table 8 (entries 6-8). (XRD=X-ray powder diffraction)

FIG. 12: CO-TPD for the Pt/TiO₂ catalyst (from Table 7, entry 1). TPD conditions: after CO chemisorption saturation at 40° C., samples were purged under flowing He (25 sccm) for 10 min, then the samples were heated up to 250° C. at a rate of 10° C./min in flowing He (25 sccm).

FIG. 13: Continuous flow reactor set up. Reaction setup consists of a downstream fixed bed reactor. Catalyst (1-5 g) was loaded inside a (⅜in) stainless steel tube, pressure was maintained using a Tescom back pressure regulator. Gases and liquid were fed using gas mass flow controllers (Brooks) and a syringe pump (ISCO Teledyne) (0-100 sccm and 0-1 mL/min, respectively). Liquid sample was collected periodically from a 50 mL collection pot downstream the reactor (50 mL, 0-10° C.). Gas sample was analyzed with a microGC instrument (Fusion

Inficon).

FIG. 14: Diffuse reflectance infrared Fourier Transform Spectroscopy (DRIFTS) spectra of fresh (blue spectrum, after post-synthesis calcination) and spent (red spectrum, collected from reactor after reaction) Pt/TiO₂ catalysts used in experiments presented in Table 7 (entries 6-8).

FIG. 15: TPO-MS profiles of the fresh and spent Pt/TiO₂ catalysts used in experiment ° C. in Table 7 (entries 6-8). Using 50 sccm of a mixture of 10 % O2/He (50 cc min⁻¹), 50 mg of the samples were ramped to 600° C. (10° C. min⁻¹). (TPO-MS=temperature-programmed oxidation with mass spectrometry)

### DETAILED DESCRIPTION OF THE INVENTION

The amine may comprise a polyamine, a tertiary amine, an alkanolamine, an aminopyridine, a diamine compound according to Formula I, or any combination thereof, the compound according to Formula I having a structure R¹(R²)N-L¹-NH—R³ where each of R¹ and R² independently is aliphatic or cycloaliphatic or R¹ and R² together with the nitrogen to which they are attached form a heterocyclic ring, L¹ is aliphatic or cycloaliphatic or L¹ and R¹ together with the nitrogen to which they are attached form a heterocyclic ring, and R³ is aliphatic, cycloaliphatic, cycloalkylalkyl or alkoxyalkyl. In certain embodiments, the amine is not an amidine, since amidines may decompose under the reaction conditions.

R¹(R²)N-L¹-NH—R³ (Formula I)

With respect to Formula I, each of R¹ and R² independently is aliphatic, preferably alkyl, such as C₁₋₆alkyl, C₁₋₄alkyl, C₁₋₃alkyl, or C₁₋₂alkyl; cycloaliphatic, preferably cycloalkyl, such as C₃₋₇cycloalkyl or C₃₋₄cycloalkyl, and may be cyclopropyl; or R¹ and R² together with the nitrogen to which they are attached, form a heterocyclic ring, such as an non-aromatic heterocyclic ring, preferably a 5- or 6-membered heterocyclic ring and optionally comprising one or more additional heteroatoms, such as 1 or 2 heteroatoms selected from oxygen, nitrogen or sulfur, and/or optionally substituted with alkyl, such as C₁₋₄alkyl. Alternatively, R¹ may form a heterocyclic moiety, with L¹, such as a non-aromatic heterocyclic moiety, preferably a 5- or 6-membered heterocyclic moiety. In such embodiments, R² is aliphatic, preferably alkyl, such as C₁₋₆alkyl, C₁₋₄alkyl, C₁₋₃alkyl, or C₁₋₂alkyl, or cycloalkyl, such as C₃₋₇cycloalkyl.

Each of R¹ and R² independently is linear alkyl or branched alkyl, such as C₁₋₆linear alkyl, or C₃₋₆branched alkyl. Exemplary linear alkyl moieties include, but are not limited to methyl, ethyl, n-propyl or n-butyl, and exemplary branched alkyl moieties include, but are not limited to, isopropyl, tert-butyl, iso-butyl, or sec-butyl. And R¹ and R² may be the same or different. In other embodiments, R¹ and R² together with the nitrogen to which they are attached form a non-aromatic heterocyclic moiety, such as morpholine, thiomorpholine, piperidine, pyrrolidine, or piperazine, optionally substituted with C₁₋₄alkyl, typically, methyl, ethyl, isopropyl, or tert-butyl.

L¹ is aliphatic, preferably alkyl, such as C₂₋₄alkyl or C₂₋₃alkyl; cycloaliphatic, preferably cycloalkyl, such as C₅₋₇cycloalkyl; or L¹ and R¹ together with the nitrogen to which they are attached form a non-aromatic heterocyclic ring, such as a 5-, 6-, or 7-membered heterocyclic, optionally comprising one or more additional heteroatoms, such as 1 or 2 heteroatoms selected from oxygen, nitrogen or sulfur. In some embodiments, L¹ is —CH₂CH₂— or —CH₂CH₂CH₂—, but in other embodiments, L¹ and R¹ together with the nitrogen to which they are attached, form a 5- or 6-membered non-aromatic heterocyclic ring, such as a piperidine or pyrrolidine ring. In some such embodiments, R² is C₁₋₆alkyl, such as C₁₋₄alkyl, C₁₋₃alkyl, or C₁₋₂alkyl, preferably methyl or ethyl.

R³ is aliphatic, cycloaliphatic, cycloalkylalkyl or alkoxyalkyl. In some embodiments, R³ is alkyl, such as C₁₋₆alkyl, C₁₋₄alkyl, C₁₋₃alkyl, or C₁₋₂alkyl; cycloalkyl, such as C₃₋₇cycloalkyl or C₃₋₄cycloalkyl; cycloalkylalkyl, such as —CH₂cycloalkyl; or alkoxyalkyl, such as C₁₋₆alkyl, C₁₋₄alkyl, C₁₋₃alkyl, or C₁₋₂alkyl substituted C₁₋₄alkoxy, C₁₋₂alkoxy, or C₃₋₆cycloalkyl. R³ may be linear or branched alkyl, and may be a linear C¹⁻⁶alkyl, C₁₋₄alkyl, C₁₋₃alkyl, or C₁₋₂alkyl or a branched C₃₋₆alkyl, C₃₋₄alkyl, or C₃alkyl. Exemplary linear and branched alkyl moieties include, but are not limited to, methyl, ethyl, n-propyl, or n-butyl, and isopropyl, isopropyl, tert-butyl, iso-butyl, or sec-butyl. In some embodiments, R³ is unsubstituted, but in other embodiments, R³ is substituted, and may be substituted with alkoxy, such as C₁₋₄alkoxy, or C₁₋₂alkoxy. Exemplary alkoxy substituents include, but are not limited to, methoxy, ethoxy, propoxy, isopropoxy or cycloalkoxy, such as cyclopropoxy.

Nonlimiting Exemplary compounds within the scope of Formula I include:

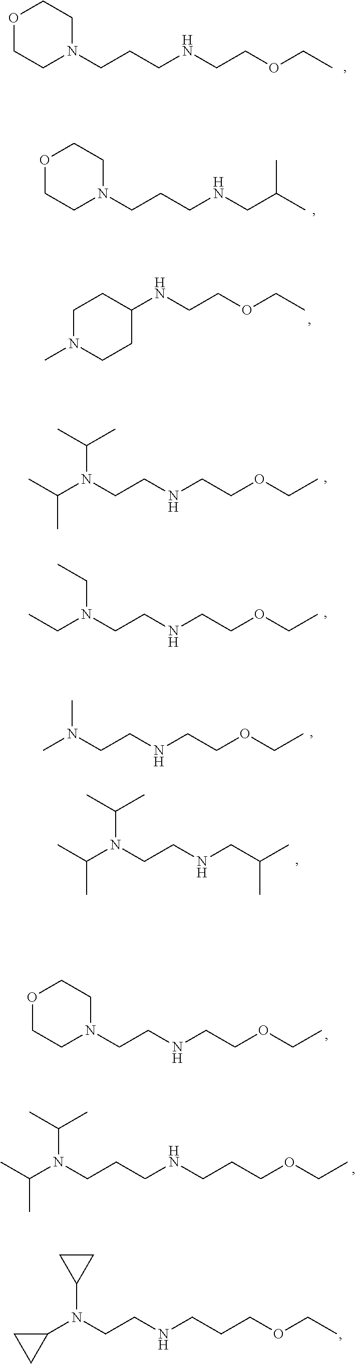

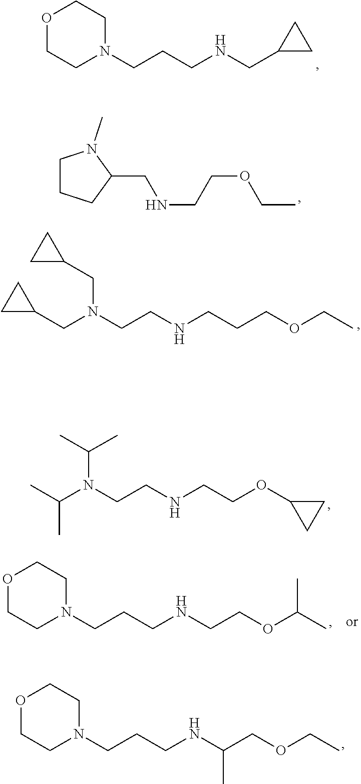

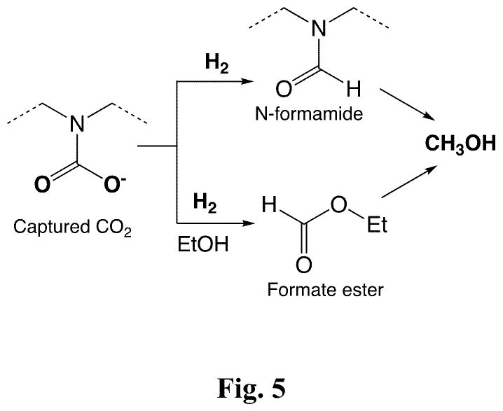

The amine sorbent can be combined with a solvent, i.e. a co-solvent or co-feed, to form a liquid sorbent solution. The co-solvent or co-feed can be any alcohol-containing species, including but not limited to aliphatic, aromatic or other polymeric or polyols, or any combination thereof. For example, a preferred co-feed is ethanol. It was observed that an ethanol co-feed facilitates reaction through formate ester intermediate, which was more selective to methanol than N-formamide intermediate. (FIG. 5)

The catalyst used in the conversion of CO₂ to products comprises a noble metal disposed on a reducible oxide support. Preferably, the catalyst comprises from 1 to 20 wt % noble metal, more preferably 1 to 10 wt %. The noble metal can be any of the noble metals or any combination of the noble metals.

The catalyst may be described by any of the properties reported herein, or within ±30% or ±20% or ±10% of any of the measurements described herein.

### Properties of Reaction

The reactions can be characterized by one or any combination of the following: an amine sorbent preferably with at least 2 wt %, or at least 5 wt %, or in the range of 3 to 15 wt % CO₂ loading; a reaction temperature of at least 140° C., or preferably at least 160° C., or in the range of 120 to 200° C.; a reaction pressure of at least 30 bar H₂, or preferably at least 50 bar H₂, or in the range of 30-100 bar H₂.

The reactions can be further characterized by one or any combination of the following: Selectivity to methanol from CO₂ is preferably at least 50%, or at least 60%, or in the range of 50 to about 95%, or 60 to about 95%. WHSV (weight hourly space velocity in units of g CO₂/g cat/h) of at least 0.005, or at least 0.010, or in the range of 0.010 to 0.50, or in the range of 0.010 to 0.20, or in the range of 0.010 to 0.10.

The catalyst and process were demonstrated to be selective towards methanol with 93% selectivity at 140° C. At 190° C., the CO₂ conversion increased from 12% to 86% when space velocity was decreased by a factor of 10. Conversion decreased from 86% to 65% over a span of approximately 40 hours.

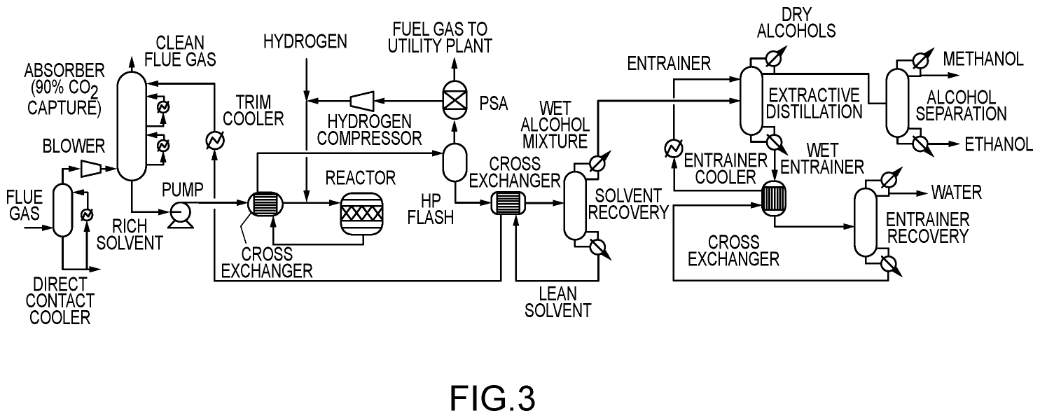

In FIG. 3, 90% CO₂ in the flue gas from a NGCC power plant is captured in the absorber by countercurrent contact with an amine sorbent solution, producing a CO₂-rich solvent. The condensed-phase CO₂-rich solvent is pumped to the desired pressure to avoid the use of a capital and energy-intensive CO₂ compressor. The pressurized CO₂-rich solvent is then heated and fed to the main reactor packed with Pt/TiO₂ catalyst at 190° C., 60 bar along with H₂. In this analysis, H₂ is produced off-site from either fossil or renewable pathway, which is outside the system boundary. It is assumed that H₂ is delivered at the conversion plant at pressure as required for H₂ transportation.^{[31]} The reactor product contains CO₂-lean solvent, unconverted H₂. methanol, C₂+ alcohols, CH₄, and light hydrocarbons. The product is first sent to a high-pressure flash drum, where the gas phase goes to the pressure swing adsorption (PSA) unit to separate unconverted H₂ from CH₄ and light hydrocarbons. The H₂ is recycled back to the reactor, while CH₄ and light hydrocarbons are used as fuel gas to supply heat and energy for other unit operations. The liquid phase from the high-pressure flash drum is sent to the solvent recovery column to separate alcohols from the CO₂-lean solvent. The lean solvent from the bottom is recycled back to the absorber for CO₂ capture. Wet alcohols from the top contain mainly methanol, ethanol, and water. Because of the existence of azeotrope between these components, extractive distillation is used for alcohols and water separation, where ethylene glycol is used as an entrainer.

### Materials and Analytical Techniques

Materials: 64 wt % Cu/Zn/Al₂O₃ was purchased from Alfa Acsar. 2-EEMPA was synthesized by following the procedure reported in the literature.^{[16a]} Metal (Pt, Pd, Ni, Cu) supported on metal oxides (TiO₂, Al₂O₃, CeO₂, SiO₂, MgO) were prepared by incipient wetness impregnation of nitrate metal precursors followed by drying (8 h at 100° C.) and calcination at 400° C. (4 h under static air). Metal oxide supports: TiO₂=Degussa P25 from Sigma Aldrich, Al₂O₃=γ-Al₂O₃ from Engelhard, CeO₂=nanopowder (<25 nm) from Sigma Aldrich, SiO₂=Davisil 646 Silica gel from Sigma Aldrich, MgO=Nanoactive (R) MgO from Nanoscale

Corporation. All other materials were purchased from commercial suppliers and used without further purification unless otherwise mentioned. All deuterated solvents were purchased from Cambridge Isotope Laboratories, Inc. 1,3,5-Trimethoxybenzene (TMB) was added as an internal standard for NMR spectroscopy.

The Cu/Zn/Al catalyst, which was derived from a hydrotalcite precursor with a Cu²⁺/Zn²⁺ atomic ratio of 2 (56% wt Cu), was prepared by a co-precipitation method at room temperature using nitrate precursors and a basic precipitation solution (2 m NaOH, 0.5 m Na₂CO₃). The precipitation product was aged (80° C.), washed, and dried overnight. The resulting precipitate hydroxide (layered hydroxide) was then calcined at 450° C. for 4 h to generate a combination of oxides.

Standard Procedure for Batch Experiments: A Parr reactor^{[34]} (100 mL) equipped with a thermocouple, pressure transducer, and reactor controller, was charged with a catalyst and CO₂ loaded capture solvent and sealed in a N₂-atmosphere glovebox. The reactor was then pressurized with H₂ (60 bar) and heated to 170° C. The reactor was cooled to room temperature, and the gas in the reactor headspace was analyzed using a 2-channel Fusion MicroGC (Inficon). The remaining excess gas was slowly released after cooling the reactor to −78° C. TMB was dissolved in acetonitrile and added as an internal standard to the reaction mixture, and a small aliquot of the sample was analyzed by ¹H and ¹³C NMR experiments in CD₃CN.

Procedure for In Situ High-Pressure and High-Temperature MAS-NMR: The MAS-NMR experiments were performed on an Agilent-Varian VNMRS NMR spectrometer equipped with a 7.05 T magnet, operating at 75.43 MHZ for the ¹³C channel and 299.969 MHz for the ¹H channel, and using a 5 mm Chemmagnetics design HXY probe. The rotors were Varian/Agilent style cavern rotors (Revolution NMR LLC), modified for high-pressure samples as described previously.^{[35]} 2-EEMPA (0.16 mmol), Cu/ZnO/Al₂O₃ or Pt/TiO₂ (8 mg), and ethanol (1.6 mmol) were transferred to a MAS-NMR rotor in a N₂-atmosphere glovebox. The rotor was charged with a ¹³CO₂ (0.28 mmol) and H₂ (0.8 mmol) at room temperature and heated to 170° C. When the set temperature was reached, the rotor was kept at this temperature while an array of ¹³C NMR spectra was collected. A 45° pulse was used. A recycle delay of 30 s and a spinning speed of 5 kHz were applied. The spectra were acquired with 64 scans and an acquisition time of 300 ms.

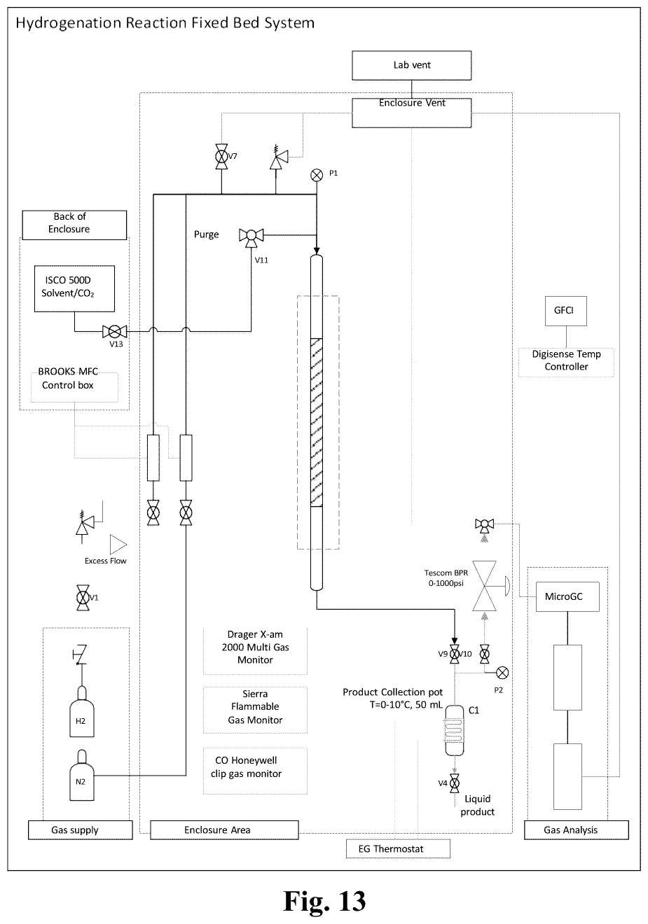

Standard Procedure for Continuous-Flow Experiments: Fixed-bed experiments were performed in a stainless-steel tubular reactor (3/8 nominal OD, 0.305 ID), the reactor wall was heated with a stainless-steel block (3 in) wrapped with a fiberglass heating tape (Omegalux) (FIG. 13). Liquid was fed to the reactor using a high-pressure Isco pump (Teledyne), and gases (H₂, N₂) were fed using mass flow controllers (Brooks). In a typical experiment, 1 g of catalyst (60-100 mesh) was loaded between quartz wool in the heated part of the reactor. Reaction conditions: pressure (60 bar) was controlled using a Tescom back pressure regulator, and temperature (120-190° C.) was controlled using a digi-sense R/S controller. After reaction, liquid products were condensed and collected in a stainless-steel 50 mL cylinder maintained at 0° C. using a recirculatory thermostat (VWR). Gas products were analyzed using a 4-channel Fusion MicroGC (Inficon), and liquid products were analyzed using liquid chromatography.

Catalysts Characterization: Fresh and spent samples of Pt/TiO₂ catalyst were studied using DRIFTS, TPO-MS, XRD, ICP, and CNHSO elemental analysis.

The DRIFTS accessory was a Harrick Praying Mantis with a reaction chamber and zinc selenide windows adapted to a Bruker IFS 66/S spectrometer. The spectra of the untreated samples were recorded with the sample at room temperature (21° C.) and at atmospheric pressure using a background with potassium bromide powder and dry nitrogen purge. The instrument resolution was 4 cm⁻¹ with an 8 mm aperture, and 1024 scans were co-added for the spectrum. A silicon carbide source was used with a KBr beam splitter and a liquid-nitrogen-cooled MCT detector. The scanner velocity was 60 kHz. The phase resolution was 32 cm⁻¹ with a Mertz phase correction. A Blackman-Harris-3-term apodization function was used with 2× zerofill. The nonlinearity correction was turned on.

TPO-MS was performed using a Micromeritics Autochem II 2920 chemisorption analyzer coupled with a Cirrus 2 Mass Spectrometer. 50 mg of sample was loaded under 50 sccm 10% O₂/He for 40 min, and without any preliminary thermal treatment, the sample was ramped up to 600° C. at a rate of 10° C. min⁻¹ and held at this temperature for 20 min. Effluent gases were monitored using mass spectrometry.

TPD and CO Chemisorption were performed using a Micromeritics Autochem II 2920 chemisorption analyzer. 100 mg of sample was loaded and was purged under flowing He (25 sccm) for 10 min. Samples were pretreated in situ at 200° C. with 10% O₂/He for 2 h, followed by reduction at 200° C. with 10% H₂/N₂ for 20 min, He purge at 200° C. for 10 min, then cooled to 40° C. for NH₃ or CO₂ or CO chemisorption. After the adsorption, samples were purged under flowing He (25 sccm) for 10 min, then the samples were heated up to 500 or 250° C. at a rate of 10° C. min⁻¹.

XRD patterns were recorded using a Philips X'pert MPD (Model PW3040/00) diffractometer with a copper anode (Ka1=0.15405 nm) and a scanning rate of 0.0013° per second between 2 h=10° and 90°. Jade 5 (Materials Data Inc., Livermore, CA) and the Powder Diffraction File database (International Center for Diffraction Data, Newtown Square, PA) were used to analyze the diffraction patterns.

The carbon, hydrogen, nitrogen, and sulfur composition of the Pt/TiO₂ catalyst before and after reaction was determined using a Thermo Fisher Flash 2000 CHNS/O organic elemental analyzer (Thermo Fisher Scientific, Inc., Waltham, MA). About 10 mg of sample was used for cach analysis.

ICP (inductively coupled plasma) was used to quantify the amount of Pt present on the Pt/TiO₂ catalyst before and after reaction. ICP used a Perkin-Elmer 3000DV with an AS90 Autosampler, which has an instrument detection limit of ≈1 ppb. About 10 mg of solid sample was prepared via microwave digestion in concentrated acid and then diluted to volume.

### Examples and Discussion

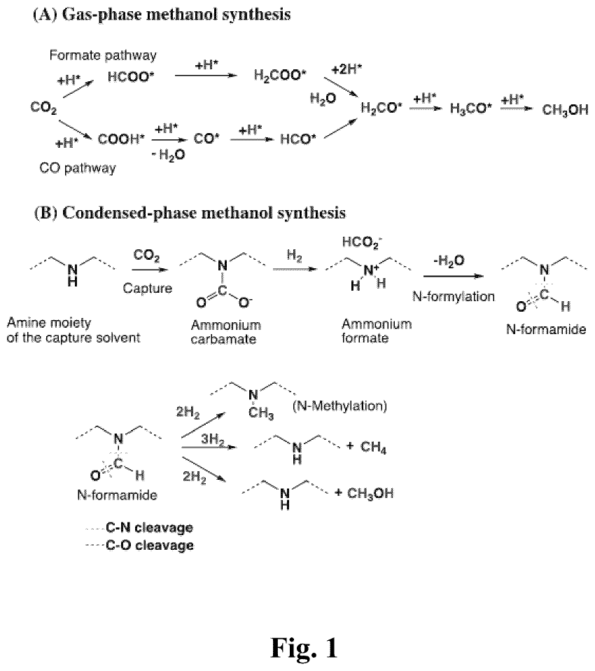

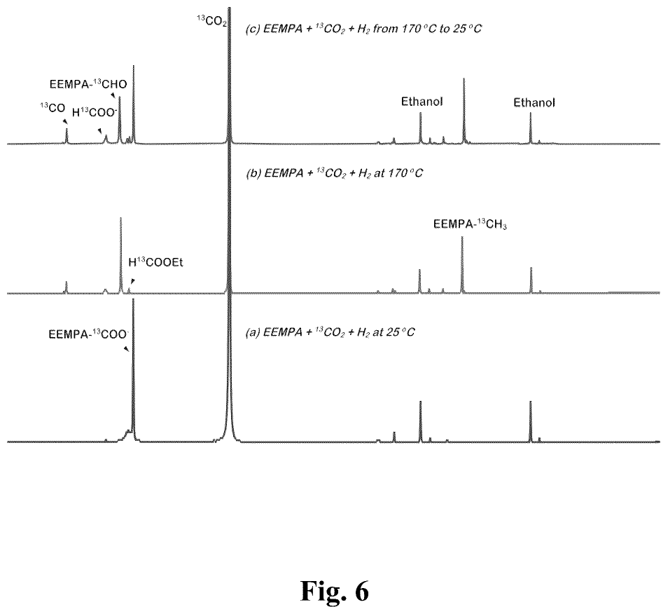

The hydrogenation of captured CO₂ was studied using EEMPA as a water-lean post combustion capture solvent. First, the CO₂ captured in EEMPA solvent (10 wt % CO₂ loading) was subjected to hydrogenation in the presence of an industrial gas phase methanol catalyst, Cu/ZnO/Al₂O₃ (entry 1, table 1). The undesired N-methylation of EEMPA was observed as a major product by ¹H NMR. To confirm N-methylation, operando NMR was performed with ¹³C-enriched CO₂ (shown in FIG. 6). At 25° C., under ¹³CO₂ and H₂ atmosphere, mostly the EEMPACOO-(carbamate) species was observed at 159.2 ppm in addition to ¹³CO₂ at 125.2 ppm. Upon heating this to 170° C., the following new species were observed: ¹³CO, EEMPA+H¹³CO₂— (formate), H¹³CO₂C₂H₅ (ester), EEMPA-¹³CHO (formamide) and EEMPA-¹³CH₃ (N-methylated EEMPA). The major species was the EEMPA-¹³CH₃ (the ¹³CH₃ signal observed at 41.7 ppm), which was formed as a result of ¹³CO₂ acting as a N-methylating agent. Similar N-methylation of amines was reported in the literature with Cu/Al₂O₃ catalyst.^{[2]} Literature reports have also shown that the rate of formation of N-methylated amine increased (and methanol decreased) at higher temperature, but the mechanism for the N-methylation is still not known.^{[3]} It is also believed that the in situ formed methanol can act as a N-methylating agent. The fact that operando NMR studies only showed traces of methanol, but excess of EEMPA-¹³CHO species, it is likely the N-methylation occurs through the formamide intermediate. The formamide can undergo C—O cleavage to form N-methylated amine or C—N cleavage to form methanol (FIG. 1). The C—N cleavage is preferred as C—O cleavage results in deactivation of the capture solvent via N-methylation.

Under the experimental conditions shown in Table 1, entry 1, the C—N cleavage selectivity was ˜25% in the case of Cu/ZnO/Al₂O₃. A CO formation was also observed via the reverse water gas shift reaction (RWGS) as a side product. The use of other amphoteric oxide supports such as hydrotalcite and ZrO₂ resulted in suppression of methanol product and only N-formamide and EEMPA-N-Me products were observed in addition to CO (Table 1, entries 2 and 4). A decreased CO₂ loading (5 wt %) improved the overall conversion and reduced RWGS reaction although the C—N cleavage selectivity remained mostly unchanged (Table 1, entry 1 vs 3). The acidic supports such as CeO₂ and TiO₂ completely suppressed both the methanol and EEMPA-Me formation, however the EEMPA-CHO was observed as a major product in both cases, which indicates that these supports in combination with Cu are not effective for the hydrogenation of the formamide intermediate under these reaction conditions (Table 1, entries 5 and 6). Overall, the surface acidity/basicity of the oxide supports considerably influenced the performance and selectivity of the catalyst (Table 1, entries 1-6).

Milstein et al. have shown that Ag/Al₂O₃ catalyst was effective for the selective C—N cleavage in the case of hydrogenation of benzamides and aliphatic amides under basic conditions.^{[4]} Because formamide is an important intermediate observed under our reaction conditions, 30 wt % Ag/Al₂O₃ was screened for the hydrogenation of captured CO₂ to improve the C—N cleavage selectivity for the hydrogenation of in-situ formed formamide intermediate, EEMPA-CHO (Table 1, entry 7). Unfortunately, Ag/Al₂O₃ was very slow for the hydrogenation of EEMPA-CHO. In addition, there was no selectivity for C—N cleavage resulting in N-methylation of EEMPA.

TABLE 1
Hydrogenation of captured CO₂ in the presence of Cu and Ag catalysts using batch reactor system.

| | | | | | | | | | | C—N |
|---|---|---|---|---|---|---|---|---|---|---|
| | | | CO2 | Product selectivity (%) | | | | | Methanol | cleavage |
| | Exp. | | conv. | | | EEMPA- | EEMPA- | | Yield | selectivity |
| Entry | No. | Catalyst | (%) | CO | CH₄ | CHO | Me | Methanol | (%) | (%) |
| 1^{a} | 218 | 64 wt % | 40.2 | 21.4 | 0.0 | 0.0 | 59.0 | 19.6 | 7.9 | 24.9 |
| | | Cu/ZaO/Al₂O₃ | | | | | | | | |
| 2^{a} | 251 | Cu/ZaO/hydrotalcite | 34.2 | 16.3 | 4.7 | 15.7 | 63.3 | 0.0 | 0.0 | 0.0 |
| 3^{b} | 283 | 64 wt % | 62.3 | 14.2 | 0.0 | 0.0 | 65.8 | 20.0 | 12.5 | 23.3 |
| | | Cu/ZnO/Al₂O₃ | | | | | | | | |
| 4^{b} | 263 | Cu/Za/ZrO₂ | 60.5 | 10.7 | 0.0 | 31.2 | 58.1 | 0.0 | 0.0 | 0.0 |
| 5^{b} | 270 | 5 wt % Cu/CeO₂ | 3.4 | 0.0 | 0.0 | 100.0 | 0.0 | 0.0 | 0.0 | — |
| 6^{b} | 281 | 35 wt % Cu/TiO₂ | 15.7 | 5.6 | 0.0 | 76.6 | 17.8 | 0.0 | 0.0 | 0.0 |
| 7^{b} | 261 | 30 wt % Ag/Al₂O₃ | 30.6 | 0.0 | 1.0 | 72.8 | 26.3 | 0.0 | 0.0 | 0.0 |

Conditions: catalyst = 200 mg, 170° C., EEMPA-5 g (CO₂ loaded EEMPA used), initial P(H₂) = 60 bar, time = 12 h,
^{a}EEMPA-(10 wt %) CO₂,
^{b}EEMPA-(6 wt %) CO₂, Cu/ZnO/Al₂O₃ = Cu (64 wt %)/ZnO(24 wt %)/Al₂O₃(5 wt %),

Traditional gas-phase CO₂ hydrogenation catalysts deactivated the solvent via N-methylation of the 2° amine moiety.
Higher conversion demonstrated at lower CO₂ loading.
The methanol selectivity compared to EEMPA N-Me was <25% in all these cases.

TABLE 2
Ethanol co-feed facilitates methanol production.

| | | | | | | N- | | CH₃OH |
|---|---|---|---|---|---|---|---|---|
| | Capture | Exp. | CO₂/H₂ | Time | Formamide | methylamine | CH₃OH | Selectivity |
| Entry | Solvent | No. | (bar) | (h) | (mmol) | (mmol) | (mmol) | (%) |
| 1 | EEMPA | 62711- | 15/45 | 12 | 0.8 | 2 | 0.65 | 24.5 |
| | | 153 | | | | | | |
| 2 | EEMPA | 62711- | 15/45 | 48 | 0.45 | 5.1 | 1.23 | 19.4 |
| | | 148 | | | | | | |
| 3 | EEMPA | 62711- | 5/55 | 48 | 0.04 | 5.85 | 1.19 | 16.9 |
| | | 147 | | | | | | |
| 4^{a} | EEMPA + | 62711- | 5/55 | 48 | traces | 1 | 2.4 | 70.6 |
| | ethanol | 150 | | | | | | |

Catalyst: Cu/ZnO/Al₂O₃ = 200 mg, 100 mL reactor, EEMPA = 23 mmol, P = 60 bar (CO₂:3H₂), T = 170° C., t = 12 h,
^{a}ethanol = 200 mmol, only the liquid phase CO₂ derived products shown.

Ethanol co-feed facilitates reaction through formate ester intermediate, which was more selective to methanol than N-formamide intermediate.
With leading PNNL post-combustion system EEMPA, process conditions tailored to form methanol with 71% selectivity.
The formation of methanol in the presence of a post combustion solvent has, we believe, been demonstrated for the first time in the presence of a heterogenous catalyst.

Several different co-feeds and process conditions were evaluated with goal to improve methanol selectivity through N-formamide intermediate.
N-formamide is harder to reduce versus formate ester
Selectivity and conversion is higher if we form the ester species
Using ethanol would complicate the processing and add costs 5 wt % Pd/Zn/Al₂O₃ was screened for the hydrogenation of captured CO₂ (Table 3, entry 1). The selectivity and performance of the 5 wt % Pd/ZnO/Al₂O₃ was comparable to that of Cu/ZnO/Al₂O₃. Upon testing the roles of Al2O3 and ZnO with 5 wt % Pd separately, it was revealed that the combination of both Al₂O₃ and ZnO were required for higher CO₂ conversion (Table 3, entries 2 and 3). In particular, the C—N cleavage selectivity was further reduced with 5 wt % Pd/Al₂O₃ catalyst. The Pd on Ga₂O₃ support predominately formed methane and some amount of CO. No N-methylation was observed in this case. A lower CO₂ loading (Table 3, entry 5) showed no improvement in the CO₂ conversion unlike the Cu/ZnO/Al₂O₃ catalyst (in Table 1, entries 1 and 3) suggesting possibly different mechanistic pathways involved irrespective of similar product distributions with Pd/Zn/Al₂O₃ and Cu/ZnO/Al₂O₃ catalysts.

The Pd on an inert support, carbon, formed EEMPA-Me with good selectivity along with small amounts of CO and methane as side products (Table 1, entry 8). A basic support such as MgO significantly improved the C—N cleavage selectivity to ˜55% (Table 1, entry 9) although the methanol yield was lower than the entry 1, Table 3 with 5wt % Pd/ZnO/Al₂O₃. Similar to the basic support, the Pd on an acidic support (CeO₂) also remarkably improved the C—N cleavage selectivity to ˜70%, resulting in significant suppression of the N-methylation.

TABLE 3
Hydrogenation of captured CO₂ in the presence of Pd catalysts using batch reactor system.

| | | | | | | | | | | C—N |
|---|---|---|---|---|---|---|---|---|---|---|
| | | | CO2 | Product selectivity (%) | | | | | Methanol | cleavage |
| | Exp. | | conv. | | | EEMPA- | EEMPA | | Yield | selectivity |
| Entry | No. | Catalyst | (%) | CO | CH₄ | N—CHO | N—Me | Methanol | (%) | (%) |
| 1^{a} | 172 | 5 wt % | 54.3 | 14.3 | 0.2 | 22.9 | 52.0 | 10.4 | 5.7 | 16.7 |
| | | Pd/ZnO/Al₂O₃ | | | | | | | | |
| 2^{a} | 255 | 5 wt % Pd/Al₂O₃ | 30.5 | 6.0 | 1.9 | 50.6 | 37.7 | 3.8 | 1.2 | 9.1 |
| 3^{a} | 174 | 5 wt % Pd/ZnO | 20.7 | 38.7 | 0.0 | 5.3 | 42.8 | 13.2 | 2.7 | 23.5 |
| 4^{a} | 249 | 5 wt % Pd/Ga₂O₃ | 9.3 | 14.0 | 86.0 | 0.0 | 0.0 | 0.0 | 0.0 | — |
| 5^{b} | 254 | 5 wt % Pd/Zn/Al₂O₃ | 55.3 | 13.3 | 2.6 | 13.7 | 60.0 | 10.4 | 5.8 | 14.8 |
| 6^{b} | 272 | 5 wt % | 3.3 | 40.2 | 20.1 | 39.7 | 0.0 | 0.0 | 0.0 | — |
| | | Pd/hydrotalcite | | | | | | | | |
| 7^{b} | 260 | 5 wt % Pd/ZnO/ | 39.8 | 14.4 | 1.1 | 9.9 | 64.5 | 10.1 | 4.0 | 13.5 |
| | | Al₂O₃ (reduced at | | | | | | | | |
| | | 400° C.) | | | | | | | | |
| 8^{b} | 269 | 5 wt % Pd/C | 18.5 | 4.4 | 2.4 | 0.0 | 93.2 | 0.0 | 0.0 | 0.0 |
| 9^{b} | 271 | 5 wt % Pd/MgO | 13.3 | 8.3 | 5.5 | 57.2 | 13.2 | 15.8 | 2.1 | 54.5 |
| 10^{b} | 266 | 5 wt % Pd/CeO₂ | 16.5 | 11.6 | 4.0 | 45.9 | 10.6 | 28.0 | 4.6 | 72.6 |
| 11^{b,c} | 267 | 5 wt % Pd/CeO₂ | 25.4 | 5.8 | 1.3 | 59.9 | 10.3 | 22.7 | 5.8 | 68.8 |

^{a}EEMPA-CO₂,
^{b}EEMPA-1/2CO₂,
^{c}ethanol, 5 wt % Pd/ZnO/Al₂O₃ = 200 mg, Pd/Zn ratio = 0.25

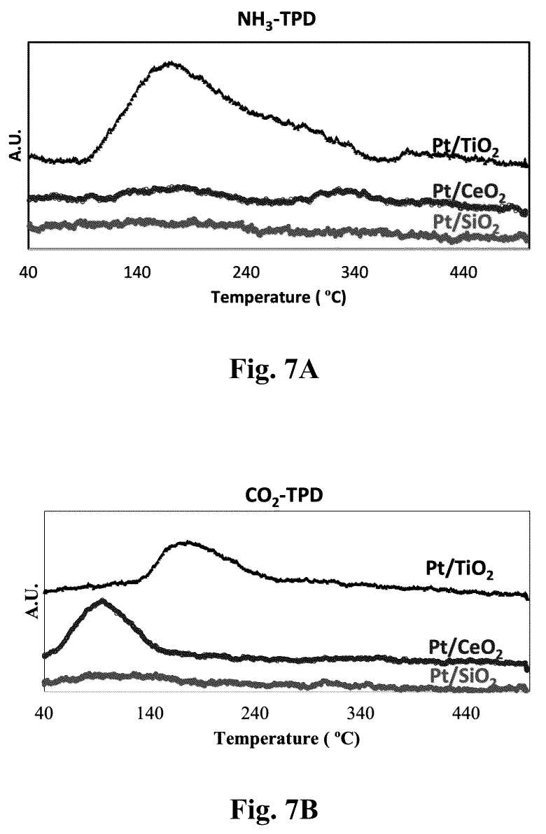

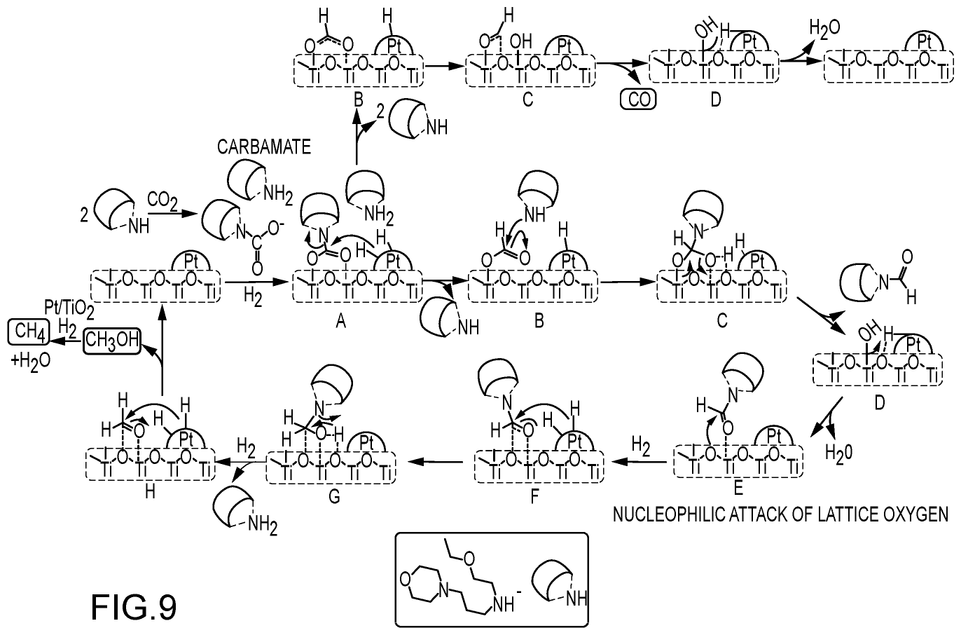

Pt/CeO₂ was screened under similar reaction conditions (Table 5, entry 1). Similar to Pd/CeO₂, Pt/CeO₂ formed methanol with high C—N bond cleavage selectivity. In addition, a considerable amount of CO was also formed in the gas phase by the rWGS reaction. The CO formation via decarbonylation of methanol has been reported in the literature.^{[21]} The TiO₂ support behaved similar to CeO₂, albeit with more methane formation and less CO production (Table 5, entry 1 vs. 2). While no methanol was formed in the case of Pt on a SiO₂ support, more CO was formed relative to Pt on a TiO₂ support (Table 1, entry 2 vs. 3). In order to understand the role of the support on the reaction mechanism, we performed CO₂-TPD (TPD=temperature programmed desorption) and NH₃-TPD to evaluate the acidic and basic nature of the Pt/TiO₂, Pt/SiO₂ and Pt/CeO₂ catalysts (FIGS. 7A and 7B, Table 4). The TPD analyses showed that Pt/TiO₂ has both acidic and basic sites, while the Pt/CeO₂ catalyst contains mostly basic sites and the Pt/SiO₂ does not have acidic or basic sites. This suggests that the basicity offered by the TiO₂ and CeO₂ supports suppresses N-methylation (see FIG. 1). Based on the experimental and TPD results, a plausible reaction mechanism for the methanol, CO and CH₄ formation from the captured CO₂ has been shown in FIG. 9. First, the captured CO₂ (i.e, carbamate) coordinates to the catalyst surface, followed by hydrogenation with hydrogen activated by Pt to form a formate species, B. The in situ formed formate species, B, formylates the amine and forms an N-formamide intermediate, E. The nucleophilic attack of the lattice oxygen of the support on the carbonyl carbon of the N-formamide intermediate, E, and subsequent hydrogenation and deamination results in the formaldehyde intermediate, H, which then gets hydrogenated again to form methanol. We believe the participation of the lattice oxygen in the nucleophilic attack of the carbon of the N-formamide intermediate is the key to the C—N cleavage selectivity. Recent reports in the literature for alcoholysis of amides have indicated that both basic and acidic sites offered by CeO₂ surfaces are required for selective C—N cleavage. However, the mechanistic understanding on the synergistic effect between Pt and the acid-base pairs offered by the supports requires further investigation.

TABLE 4
Acidity and basicity of 5 wt % Pt
catalysts using NH₃ and CO₂ TPD.

| | | Acidity | Basicity |
|---|---|---|---|
| | | NH₃-TPD | CO₂-TPD |
| Entry | Catalysts | (μmol/g) | (μmol/g) |
| 1 | 5 wt % Pt/TiO₂ | 24.4 | 5.6 |
| 2 | 5 wt % Pt/CeO₂ | 2.7 | 6.1 |
| 3 | 5 wt % Pt/SiO₂ | n.d | n.d |

100 mg of sample loaded and purged under flowing He (25 sccm) for 10 min. Samples were pretreated in situ at 200° C. with 10% O₂/He for 2 h, followed by reduction at 200° C. with 10% H₂/N₂ for 20 min, He purge at 200° C. for 10 min, then cooled to 40° C. for NH₃ or CO₂ chemisorption. After the adsorption, samples were purged under flowing He (25 sccm) for 10 min, then the samples were heated up to 500° C. at a rate of 10° C./min.

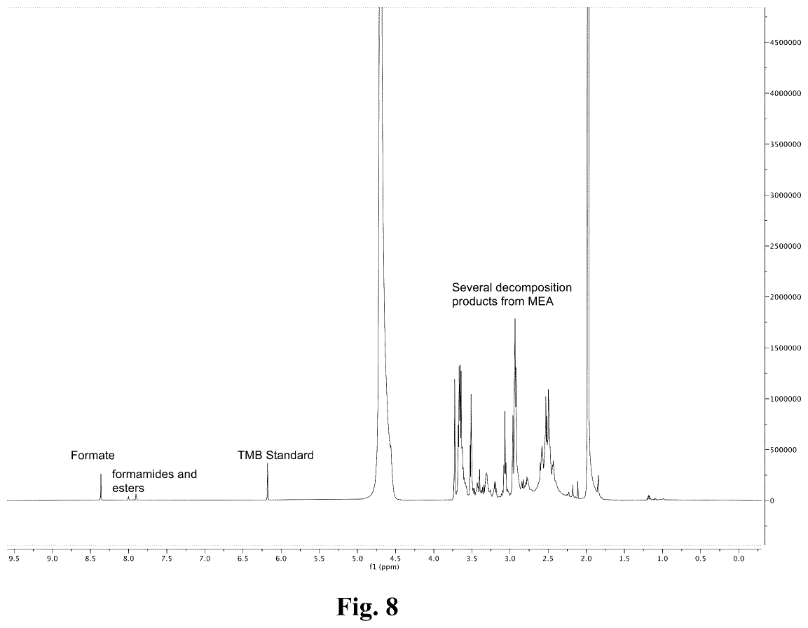

Because of the favorable chemical stability of TiO₂ in organic solvents, its strong metal-support interactions, and its acid-base properties, the Pt/TiO₂ system was explored further with different process conditions. The addition of ethanol as a co-solvent was also evaluated and improved the yields of the 2-EEMPA-CHO intermediate and methanol and decreased the yields of side products, CO, and methane (Table 5, entry 4). However, from the economic feasibility point of view, it is beneficial to avoid using ethanol as the co-solvent in the system. Because ethanol used for CO₂ conversion needs to be separated from the conversion products as well as from the carbon capture solvent, before recycling the carbon capture solvent to the CO₂ absorber. These separations are energy- and capital-intensive involving large distillation columns, big reboilers, and additional process units to break azeotropic between ethanol and water by-product. At low reaction temperatures (e.g., 150° C.) and with short reaction times, methanol was formed with high selectivity, and 100% selectivity toward C—N bond cleavage was observed (Table 5, entries 5 and 7). Table 5, entry 5 clearly shows that N-methylation and methanol formation do not occur in parallel with Pt/TiO₂-instead, the methanol is likely acting as an N-methylation source. Thus, by avoiding the longer contact time between the methanol and catalyst, N-methylation can be avoided. On the other hand, at lower H₂ pressure, the methanol yield and C—N bond cleavage selectivity were decreased (Table 5, entry 6). The commonly employed post-combustion solvent 30 wt % MEA was also evaluated for the hydrogenation of captured CO₂ with a Pt/TiO₂ catalyst (Table 5, entry 8). ¹H NMR analysis revealed the formation of mostly MEA-formate, MEA-N-CHO species, and several MEA decomposition products (FIG. 8). This shows that the current approach of combined capture and conversion to methanol is not practical when using the conventional MEA solvent, in large part because the reaction involves elimination of water, and the formation of water is hindered in aqueous solution (FIG. 1).

TABLE 5
Hydrogenation of captured CO₂ in the presence of Pt-supported
catalysts using batch reactor system.

| | | | | | | | | | C—N |
|---|---|---|---|---|---|---|---|---|---|
| | | CO₂ | Product selectivity (%) | | | | | Methanol | cleavage |
| | | conv. | | | EEMPA- | EEMPA | | Yield | selectivity |
| Entry | Catalyst | (%) | CO | CH₄ | N—CHO | N—Me | Methanol | (%) | (%) |
| 1 | 5 wt % Pt/CeO₂ | 29.7 | 49.5 | 3.3 | 11.4 | 11.4 | 24.4 | 7.2 | 68.0 |
| 2 | 5 wt % Pt/TiO₂ | 29.1 | 31.9 | 19.8 | 9.0 | 12.3 | 27.0 | 7.9 | 68.7 |
| 3 | 5 wt % Pt/SiO₂ | 23.9 | 77.4 | 0.0 | 15.3 | 7.3 | 0.0 | 0.0 | 0.0 |
| 4^{a} | 5 wt % Pt/TiO₂ | 42.4 | 15.6 | 7.8 | 37.7 | 11.7 | 27.2 | 11.5 | 69.8 |
| 5^{b} | 5 wt % Pt/TiO₂ | 11.5 | 35.8 | 25.6 | traces | traces | 38.7 | 4.5 | 100.0 |
| 6^{d} | 5 wt % Pt/TiO₂ | 15.9 | 0.9 | 25.9 | 18.1 | 26.4 | 28.6 | 4.5 | 52.0 |
| 7^{e} | 5 wt % Pt/TiO₂ | 12.2 | 25.5 | 22.4 | 15.0 | traces | 37.1 | 4.5 | 100.0 |
| 8^{f} | 5 wt % Pt/TiO₂ | 44.7 | 5.8 | 1.6 | 38.1 | 5.9 | 7.6 | 3.4 | 56.5 |
| 9^{c} | 5 wt % Pt/TiO₂ | 19.3 | 29.8 | 16.9 | 10.6 | 10.1 | 32.6 | 6.3 | 76.4 |

Conditions: catalyst = 200 mg, 170° C., EEMPA-5 g (CO₂ loaded EEMPA used, 6 wt % CO₂ loading), initial P(H₂) = 60 bar, time = 12 h,
^{a}ethanol,
^{b}3 h,
^{c}10 wt % CO₂,
^{d}30 bar H₂,
^{e}150° C.,
^{f}30 wt % MEA was used as a capture solvent; mostly MEA-formate and MEA-N-formamide species were observed; MEA decomposition products were also observed under the reaction conditions.

In additional batch reactor studies, the TiO₂ support was screened with different metals such as Ni, Ru, Cu, and Pd to understand the role of Pt in selective C—N bond cleavage (Table 6). In the reactions with Ni and Ru, methane was formed with high selectivity and no methanol was observed (Table 6, entries 2 and 3). To the best of our knowledge, this is the first example of methane formation from captured CO₂ using a non-noble metal catalyst in the presence of a capture solvent. No methanol was observed in the presence of a Cu/TiO₂ catalyst, but only N-methylation was observed via undesired C—O bond cleavage (Table 5, entry 4, also shown in Table 1, entry 6). High Cu content on the TiO₂ support could have limited the availability of the TiO₂ support for the C—N cleavage selectivity. It is also important to note that low Cu content (5 wt %) in Cu/CeO₂ catalyst was not sufficient to hydrogenate the EEMPA-CHO intermediate (Table 1, entry 5).

When Pd was used in place of Pt, the Pd/TiO₂ was less active for the final formamide hydrogenation step (Table 3, entry 5) because Pd has a low concentration of hydrogen atoms on

the surface compared to Pt.^{[24]} Therefore, the results in Tables 5 and 6 clearly show that the combination of Pt and TiO₂ or CeO₂ supports is best suited for methanol production with reduced or negligible deactivation of the capture solvent.

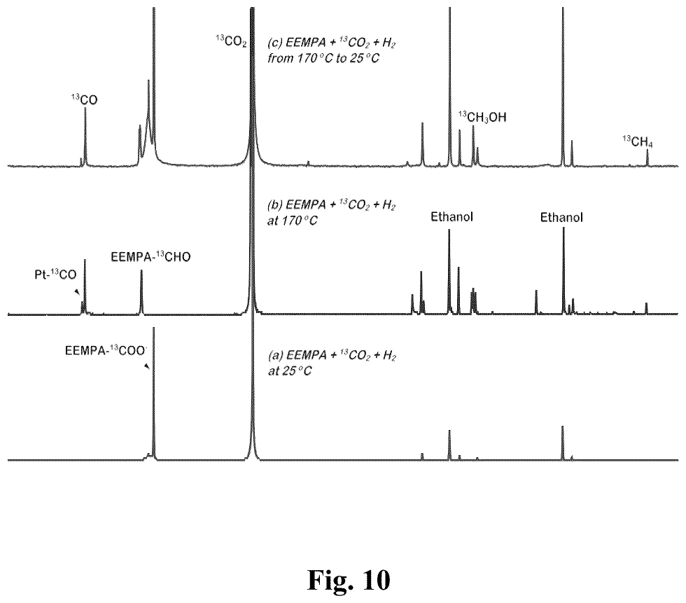

Operando ¹³C NMR was performed with ¹³C-enriched CO₂ in the presence of a Pt/TiO₂ catalyst to understand the reaction pathway in the condensed phase and confirm the absence of N-methylation. The NMR analysis showed ¹³CO, 2-EEMPA-¹³CHO, ¹³CH₃OH, and ¹³CH₄ as predominant species at 183.1, 164.4, 49.1, and −11 ppm, respectively, at 170° C. Simultaneous formation of all these species suggests that methanol can form via both CO and N-formamide intermediates. CO could also form from the decarbonylation of methanol. It is very important to note that no detectable amount of the undesired 2-EEMPA-¹³CH₃ species was observed, unlike the Cu/ZnO/Al₂O₃ catalyst under similar reaction conditions (FIG. 6 vs. FIG. 10).

TABLE 6
Hydrogenation of captured CO₂ in the presence of metals with TiO₂ supports using batch reactor system.

| | | | | | | | | | | | C—N |
|---|---|---|---|---|---|---|---|---|---|---|---|
| | | | CO₂ | Product selectivity (%) | | | | | | Methanol | cleavage |
| | Exp. | | conv. | | | EEMPA- | EEMPA | | | Yield | selectivity |
| Entry | No. | Catalyst | (%) | CO | CH₄ | N—CHO | N—Me | Methanol | C₂₊ | (%) | (%) |
| 1 | 274 | 5 wt % Pt/TiO₂ | 29.1 | 31.9 | 19.8 | 9.0 | 12.3 | 27.0 | 0 | 7.9 | 68.7 |
| 2 | 287 | 35 wt % Ni/TiO₂ | 77.9 | 0.0 | 91.4 | 6.4 | 0.0 | 0.0 | 2.2 | 7.2 | 68.0 |
| 3 | 300 | 5 wt % Ru/TiO₂ | >99 | 0.0 | >90 | 0.0 | 0.0 | 0.0 | >10 | 0.0 | — |
| 4 | 281 | 35 wt % Cu/TiO₂ | 15.7 | 5.6 | 0.0 | 76.6 | 17.8 | 0.0 | 0.0 | 0.0 | 0.0 |
| 5 | 273 | 5 wt % Pd/TiO₂ | 19.9 | 3.7 | 16.2 | 80.1 | 0.0 | 0.0 | 0.0 | — | — |

Conditions: catalyst = 200 mg, 170° C., EEMPA-5 g (CO₂ loaded EEMPA used, 6 wt % CO₂ loading), initial P(H₂) = 60 bar, time = 12 h

The most promising catalyst formulation Pt/TiO₂ identified in batch reactor studies was then evaluated under continuous-flow operation for a range of operating temperatures and space velocities using CO₂ captured in neat 2-EEMPA solvent (Table 7). When operating at 140° C., the CO₂ conversion was very low (2%) (Table 7, entry 1). However, the catalyst was highly selective toward methanol, with 93% selectivity. Interestingly, the remaining product was primarily propanol, with 7% selectivity. At higher temperatures (170 and 190° C.), ethanol and smaller amounts of butanol were also detected (Table 7, entries 2-8). While the mechanism for their formation is not yet clear, we can learn from decades of research on higher alcohol synthesis from the hydrogenation of CO and CO₂.^{[25.26]} For example, the synergistic effects between metal nanoparticles and the underlying TiO₂ support, especially the anatase crystal phase with abundant oxygen vacancies, have been reported to facilitate ethanol formation from CO₂ and H₂.^{[27]} Thus, this is a very important finding because the production of higher alcohols from syngas and/or CO₂ has been the subject of significant research in the area of conventional thermochemical catalysis. Furthermore, no CO or CO₂ was observed in the gas phase for any of the conditions evaluated. Confirmation that the reaction is primarily occurring in the condensed phase is significant, because any loss of carbon in the form of gas-phase CO or CO₂ would require additional energy and cost for recapture and conversion.

Also, when the operating temperature was increased from 140 to 190° C. (Table 7, entry 5), the CO₂ conversion modestly increased from 2% to 12%. However, the methanol selectivity was reduced from 93% to 78%. When higher alcohols were formed in addition to methanol, the generation of methane with 15% selectivity was also observed. While methane is considered an undesired side product in alcohol production reaction, the production of methane could have its advantages as a CO₂-neutral fuel. When operating at 190° C., the space velocity was decreased by a factor of 10 and the conversion increased from 12% to 86% (Table 5, entries 4 and 6). However, methanol selectivity decreased from 78% to 52% because of increasing methane and ethane formation.

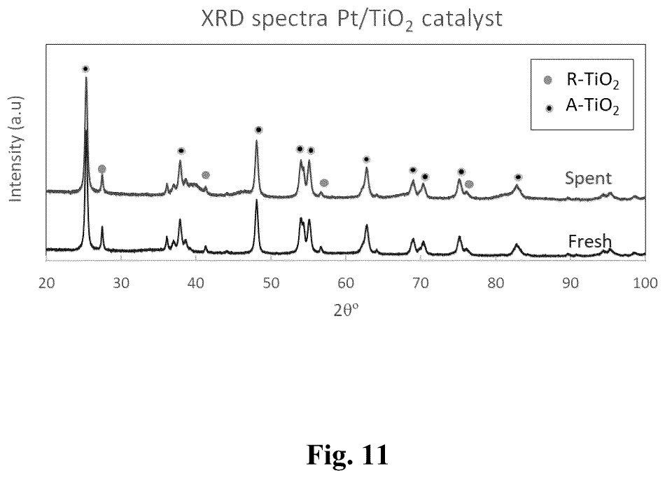

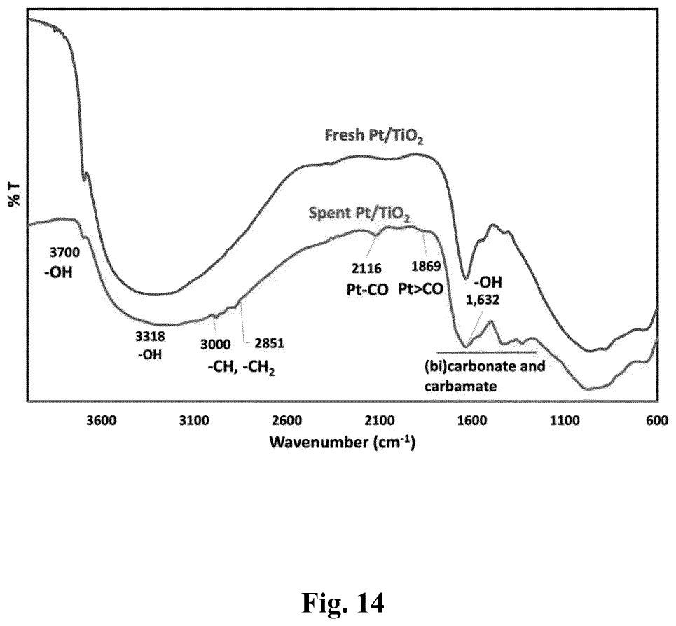

Catalytic performance results in Table 7 (entries 6-8) show how the activity of Pt/TiO₂ decreases over time (CO₂ conversion steadily drops from 85.7% to 65.2% over 80 h of reaction). To understand possible underlying deactivation mechanisms, we performed postmortem characterization of the catalyst used in this reaction using diffuse reflectance infrared Fourier transform spectroscopy (DRIFTS), temperature-programmed oxidation with mass spectrometry (TPO-MS), X-ray powder diffraction (XRD), and inductively coupled plasma (ICP) and compared the results with those of the fresh catalyst. DRIFTS of the fresh and spent catalysts showed the presence of adsorbed water (FIG. 14). The spent catalyst also showed alkane C—H stretching bands between 3000 and 2800 cm⁻¹, which corresponds to the capture solvent (EEMPA). The main infrared features of the spent catalyst are the presence of CO adsorbed on Pt and of carbonate-like species in different adsorption modes (note that these carbonate-like species are also present in the fresh catalyst sample). The spent catalyst showed the presence of a terminal Pt—CO (2116 cm⁻¹) and a weak band at 1873 cm⁻¹ for bridged Pt—CO. Multiple bands appeared in the carbonate region between 1700 and 1200 cm⁻¹, corresponding to the carbonates, carbamates, and bicarbonates.^{[28]} Both species (CO and carbonates) have been described in the literature as reasons for metal/support catalyst deactivation—in the case of CO due to a reduction of active sites on the Pt surface and in the case of carbonates due to a reduced number of interface Pt—TiO₂ active sites where they are preferentially formed.^{[28]} Physical changes to the catalyst (e.g., sintering/leaching of platinum particles) cannot be discarded, despite the mild conditions used. There was no appreciable change in the Pt dispersion between the fresh and spent catalysts based on the CO chemisorption (Table 8), suggesting no Pt sintering during the reaction. The presence of organic residue on the spent catalyst makes ICP (for Pt) and elemental analysis (C, H, N, S) quantification difficult or inconclusive. The Pt content of the fresh and spent Pt/TiO₂ catalysts was 5.6% and 4.5%, respectively. The capture solvent absorbed on the spent catalyst could be the reason for the low Pt content (and high C, H, N content) in the spent catalyst. The Pt crystallite size as determined by XRD remained below the detection limit. Further, XRD did not indicate any change in the TiO₂ support predominantly anatase phase after reaction. The XRD and elemental analysis (CHNSO) results are presented in FIG. 11.

TABLE 7
Hydrogenation of captured CO₂ over Pt/TiO₂ catalyst over a range of operating
temperatures and space velocities using a continuous flow reactor system.

| | Reaction | CO₂ | | | | | | | | |
|---|---|---|---|---|---|---|---|---|---|---|
| | temperature | Conversion | WHSV | TOS | Selectivity [mol C %] | | | | | |
| Entry | [° C.] | [%] | g_{CO2}/g_{cat}/h | [h] | MeOH | EtOH | PrOH | BuOH | CH₄ | C₂H₆ |
| 1 | 140 | 2.2 | 0.15 | — | 92.7 | 0.0 | 7.3 | 0.0 | 0.0 | 0.0 |
| 2 | 170 | 7.7 | 0.15 | — | 66.5 | 4.3 | 2.5 | 0.7 | 26.0 | 0.0 |
| 3 | 170 | 29.1 | 0.015 | — | 57.0 | 4.5 | 0.8 | 1.4 | 26.7 | 8.7 |
| 4 | 190 | 11.8 | 0.15 | — | 78.0 | 4.3 | 0.0 | 2.5 | 15.1 | 0.0 |
| 5 | 190 | 26.9 | 0.075 | — | 63.6 | 4.6 | 0.2 | 1.9 | 26.4 | 3.3 |
| 6 | 190 | 85.7 | 0.015 | 40 | 51.5 | 9.7 | 0.6 | 1.9 | 27.1 | 9.3 |
| 7 | 190 | 75.9 | 0.015 | 60 | 50.2 | 8.6 | 0.7 | 2.0 | 29.2 | 9.3 |
| 8 | 190 | 65.2 | 0.015 | 80 | 46.0 | 8.0 | 1.1 | 4.7 | 29.8 | 10.5 |

Liquid feed: captured CO₂ in EEMPA solvent (5 wt % CO₂) over 5 wt % Pt/TiO₂ catalyst. Reaction conditions: 1.0 g catalyst, 870 psig; Gas feed: (0.05, 0.025, 0.005 mL/min). 38 sccm H₂, 5 sccm N₂. Change in WHSV is achieved by changing the liquid feed flow. Catalyst was pretreated in situ overnight at 120° C. under reducing flow (50 sccm 10% H₂ in N₂).

TABLE 8
Pt dispersion characterization of 5 wt % Pt
catalysts using CO pulse chemisorption.

| | | CO Chemisorption | CO Chemisorption |
|---|---|---|---|
| Entry | Catalysts | Surface Pt (μmol/g) | Pt dispersion |
| 1 | 5 wt % Pt/TiO₂ | 72.5 | 28.6% |
| 2 | 5 wt % Pt/TiO₂ (spent) | 74.0 | 28.9% |
| 3 | 5 wt % Pt/CeO₂ | 63.4 | 25.1% |
| 4 | 5 wt % Pt/SiO₂ | 77.35 | 30.2% |

Analytical conditions: 100 mg of sample was purged under flowing He (25 sccm) for 10 min. Samples were pretreated in situ at 200° C. with 10% O₂/He for 2 h, followed by reduction at 200° C. with 10% H₂/N₂ for 20 min, He purge at 200° C. for 10 min, then cooled to 40° C. for CO chemisorption.

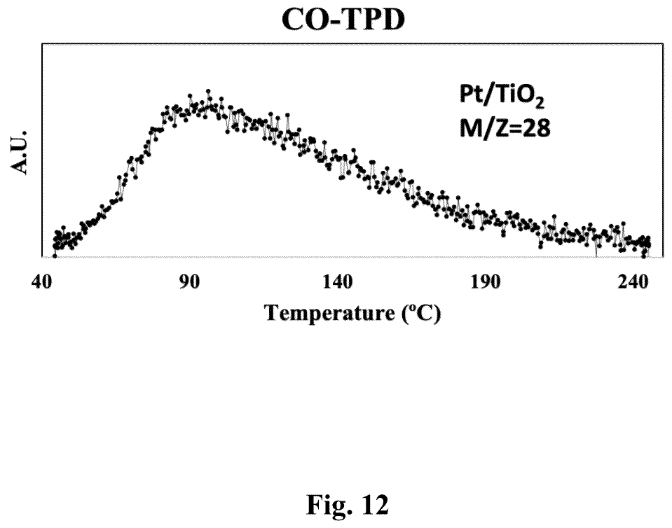

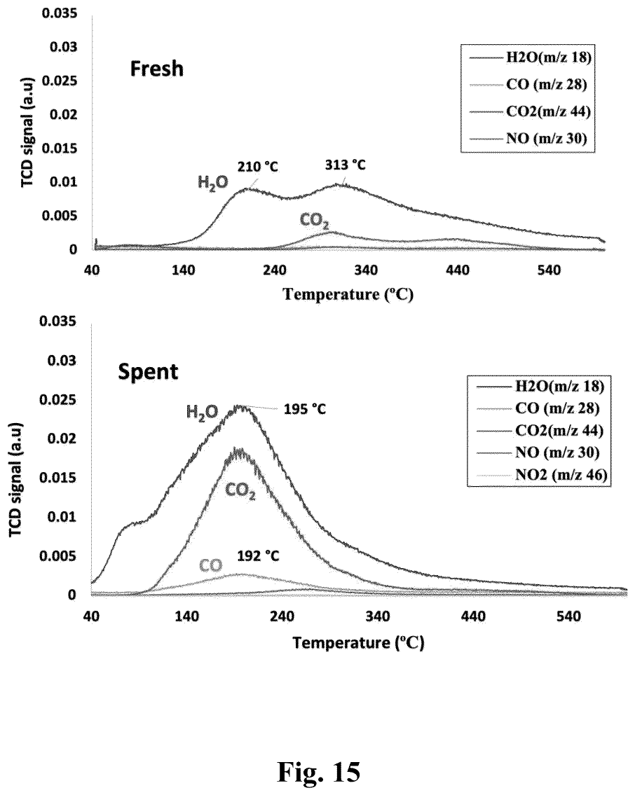

The TPO-MS analysis of the fresh Pt/TiO₂ catalyst showed signals for the loss of water between 150 and 500° C. (FIG. 15). The spent catalyst showed the presence of water, CO₂, and a small amount of CO. The metal-bound CO is possibly formed from the rWGS reaction. The following could be potential CO₂ sources: 1) the 2-EEMPA carbamate species bound to the Pt, which can release CO₂ with an increase in temperature (2-EEMPA-CO_{2—} to 2-EEMPA+CO₂), 2) surface bicarbonate species formed upon the reaction of CO₂ or 2-EEMPA carbamate with water. The bicarbonate species can decompose to produce CO₂, water, and carbonate species (2HCO₃₋ to CO₂+H₂O+CO₃²⁻), 3) oxidation of adsorbed organic species such as HCO₂⁻, 2-EEMPA, 2-EEMPA-CHO and 2-EEMPA-CH₃. The concurrent formation of water and CO₂ points to the oxidation of these organic species, 4) oxidation of adsorbed CO— under TPO conditions (10% O₂/He), the surface-bound CO species could oxidize to give CO₂ (2CO+O₂ to 2CO₂). In addition, the TPO profile of the spent catalyst (FIG. 15) shows that desorption and oxidation of adsorbed species occur at moderate temperatures (<300° C.) and that CO gets desorbed at temperatures slightly higher than the reaction temperature (190° C.). This relatively strong (and extended) CO adsorption is consistent with literature reports, which indicate this as the main reason for Pt deactivation at these mild reaction temperatures (<200° C.) and for weaker CO—Pt adsorption at higher temperatures.^{[28]} Solvent adsorbed on the catalyst (3000-2800 cm⁻¹ infrared bands in FIG. 14) decomposes also at moderate oxidation temperatures (NO peak max at 290° C. in FIG. 15). Both DRIFTS and TPO-MS analyses suggest that CO and carbonates are blocking the active sites of the spent catalyst. However, operando ¹³C MAS-NMR showed only the presence of metal-bound CO under the reaction conditions (at 170° C.) and that the carbonate/carbamate/bicarbonate species are formed only at room temperature (FIG. 15). CO-TPD was performed on the CO chemisorbed Pt/TiO₂ catalyst to further evaluate strong CO adsorption. CO-TPD results suggest that CO is strongly bound to the catalyst surface (CO—Pt) even at the reaction temperature of 190° C. (FIG. 12). Overall, based on DRIFTS, in situ ¹³C MAS-NMR, and TPO-MS, we conclude that the catalyst is most likely poisoned by the strong adsorption of CO on Pt. Regeneration/reactivation is possible at relatively moderate oxidation/desorption temperatures.

### References

[1] https://www.un.org/en/climatechange/net-zero-coalition (accessed: September 2022).
[2] a) A. Al-Mamoori, A. Krishnamurthy, A. A. Rownaghi, F. Rezaci, Energy Technol. 2017, 5, 834; b) P. Gabrielli, M. Gazzani, M. Mazzotti, Ind. Eng. Chem. Res. 2020, 59, 7033; c) J. Leclaire, D. J. Heldebrant, Green Chem. 2018, 20, 5058; d) E. C. Ra, K. Y. Kim, E. H. Kim, H. Lee, K. An, J. S. Lec, ACS Catal. 2020, 10, 11318; c) M. Bui, C. S. Adjiman, A. Bardow, E. J. Anthony, A. Boston, S. Brown, P. S. Fennell, S. Fuss, A. Galindo, L. A. Hackett, J. P. Hallett, H. J. Herzog, G. Jackson, J. Kemper, S. Krevor, G. C. Maitland, M. Matuszewski, I. S. Metcalfe, C. Petit, G. Puxty, J. Reimer, D. M. Reiner, E. S. Rubin, S. A. Scott, N. Shah, B. Smit, J. P. M. Trusler, P. Webley, J. Wilcox, N. M. Dowell, Energy Environ. Sci. 2018, 11, 1062; f) C. Hepburn, E. Adlen, J. Beddington, E. A. Carter, S. Fuss, N. M. Dowell, J. C. Minx, P. Smith, C. K. Williams, Nature 2019, 575, 87; g) J. Tollefson, Nature 2022, https://doi.org/10.1038/d41586-022-00585-7; h) M. Peplow, Nature 2022, 603, 780.
[3] National Academies of Sciences, Engineering, and Medicine, Negative Emissions Technologies and Reliable Sequestration: A Research Agenda, The National Academies Press, Washington DC 2019.
[4] https://www.iea.org/commentaries/is-carbon-capture-too-expensive (accessed: September 2022).
[5] X. Wang, C. Song, Front. Energy Res. 2020, 8, 560849.
[6] https://www.iea.org/reports/ccus-in-clean-energy-transitions (accessed: September 2022).
[7] D. W. Keith, G. Holmes, D. St. Angelo, K. Heidel, Joule 2018, 2, 1573.
[8] a) D. Aaron, C. Tsouris, Sep. Sci. Technol. 2005, 40, 321; b) P. Luis, Desalination 2016, 380, 93; c) D. J. Heldebrant, P. K. Koech, V. A. Glezakou, R. Rousseau, D. Malhotra, D. C. Cantu, Chem. Rev. 2017, 117, 9594; d) D. J. Heldebrant, J. Kothandaraman, in Carbon Capture and Storage (Eds: M. Bui, N. M. Dowell), The Royal Society of Chemistry, London 2020, p. 36-68.
[9] a) D. B. Lao, B. R. Galan, J. C. Linchan, D. J. Heldebrant, Green Chem. 2016, 18, 4871; b) J. Kothandaraman, R. A. Dagle, V. L. Dagle, S. D. Davidson, E. D. Walter, S. D. Burton, D. W. Hoyt, D. J. Heldebrant, Catal. Sci. Technol. 2018, 8, 5098; c) S. Xic, W. Zhang, X. Lan, H. Lin, ChemSusChem 2020, 13, 6141; d) S. Kar, A. Goeppert, G. K. S. Prakash, Acc. Chem. Res. 2019, 52, 2892; c) N. M. Rezaycc, C. A. Huff, M. S. Sanford, J. Am. Chem. Soc. 2015, 137, 1028; f) J. Kothandaraman, D. J. Heldebrant, Green Chem. 2020, 22, 828; g) M. Scott, B. Blas Molinos, C. Westhues, G. Francio, W. Leitner, ChemSusChem 2017, 10, 1085; h) J. Kothandaraman, J. Zhang, V.-A. Glezakou, M. T. Mock, D. J. Heldebrant, J. CO2 Util. 2019, 32, 196; i) J. W. Maina, J. M. Pringle, J. M. Razal, S. Nunes, L. Vega, F. Gallucci, L. F. Dumec, ChemSusChem 2021, 14, 1805; j) J. B. Jakobsen, M. H. Ronne, K. Daasbjerg. T. Skrydstrup, Angew. Chem., Int. Ed. 2021, 60, 9174; k) J. Bi, P. Hou, F. W. Liu, P. Kang. ChemSusChem 2019, 12, 2195; l) M. Yadav, J. C. Linchan, A. J. Karkamkar, E. van der Eide, D. J. Heldebrant, Inorg. Chem. 2014, 53. 9849; m) R. Sen, C. J. Koch, A. Goeppert, G. K. S. Prakash, Chem-SusChem 2020, 13, 6318; n) E. A. K. Wilson, S. C. Eady, T. Silbaugh, L. T. Thompson, M. A. Barteau, J. Catal. 2021, 404. 977; o) S. Ni, J. Zhu, R. Roy, C.-J. Li, R. B. Lennox, Green Chem. 2021, 23, 3740; p) M. Lu, J. Zhang, Y. Yao, J. Sun, Y. Wang, H. Lin, Green Chem. 2018, 20, 4292; q) J. R. Khusnutdinova, J. A. Garg. D. Milstein, ACS Catal. 2015, 5, 2416; r) N. D. McNamara, J. C. Hicks, ChemSusChem 2014, 7, 1114; s) C. Reller, M. Poge, A. Lissner, F. O. Mertens, Environ. Sci. Technol. 2014, 48, 14799; t) S. Overa, T. G. Feric, A.-H. A. Park, F. Jiao, Joule 2021, 5, 8; u) R. Sen, C. J. Koch, V. Galvan, N. Entesari, A. Goeppert, G. K. S. Prakash, J. CO2 Util. 2021, 54, 101762; v) D. Wei, H. Junge, M. Beller, Chem. Sci. 2021, 12, 6020; w) D. Wei, R. Sang, A. Moazezbarabadi, H. Junge, M. Beller, JACS Au 2022, 2, 1020; x) S. Kar, A. Goeppert, J. Kothandaraman, G. K. S. Prakash, ACS Catal. 2017, 7, 6347.
[10] a) M. Scott, C. G. Westhues, T. Kaiser, J. C. Baums, A. Jupke, G. Francio, W. Leitner, Green Chem. 2019. 21. 6307; b) J. Kothandaraman, D. J. Heldebrant, RSC Adv. 2021. 10, 42557; c) A. Sternberg, C. M. Jens, A. Bardow, Green Chem. 2017, 19, 2244; d) J. Klankermayer, W. Leitner, Science 2015, 350, 629; c) Q. Liu, L. Wu, R. Jackstell, M. Beller, Nat. Commun. 2015, 6, 5933; f) T. Sakakura, J. C. Choi, H. Yasuda, Chem. Rev. 2007, 107, 2365; g) G. A. Olah, A. Goeppert, G. K. Prakash, J. Org. Chem. 2009, 74, 487; h) J. Klankermayer, S. Wesselbaum, K. Beydoun, W. Leitner, Angew. Chem., Int. Ed. 2016, 55, 7296; i) C. Song, Catal. Today 2006, 115, 137. j) G. A. Olah, G. K. Prakash, A. Goeppert, J. Am. Chem. Soc. 2011, 133, 12881; k) S. van Bavel, S. Verma, E. Negro, M. Bracht, ACS Energy Lett. 2020, 5, 2597; l) Z. Zhang. T. Wang. M. J. Blunt, E. J. Anthony, A.-H. A. Park, R. W. Hughes, P. A. Webley, J. Yan, Appl. Energy 2020, 278, 115627.
[11] a) J. Kothandaraman, J. Saavedra Lopez, Y. Jiang, E. D. Walter, S. D. Burton, R. A. Dagle, D. J. Heldebrant, ChemSusChem 2021, 14, 4812; b) D. J. Heldebrant, J. Kothandaraman, N. M. Dowell, L. Brickett, Chem. Sci. 2022, 13, 6445.
[12] J. Kothandaraman, A. Goeppert, M. Czaun, G. A. Olah, G. K. Prakash, J. Am. Chem. Soc. 2016. 138, 778.
[13] a) S. Kanuri, S. Roy, C. Chakraborty, S. P. Datta, S. A. Singh, S. Dinda, Int. J. Energy Res. 2022, 46, 5503; b) M. D. Porosoff, B. Yan, J. G. Chen, Energy Environ. Sci. 2016, 9, 62; c) S. Kattel, P. J. Ramirez, J. G. Chen, J. A. Rodriguez, P. Liu, Science 2017, 355, 1296.
[14] a) S. Kar, J. Kothandaraman, A. Goeppert, G. K. S. Prakash, J. CO2 Util. 2018, 23, 212; b) S. Kar, R. Sen, J. Kothandaraman, A. Goeppert, R. Chowdhury, S. B. Munoz, R. Haiges, G. K. S. Prakash, J. Am. Chem. Soc. 2019, 141. 3160.
[15] Y. Jiang, P. M. Mathias, C. J. Freeman, J. A. Swisher, R. F. Zheng, G. A. Whyatt, D. J. Heldebrant, Int. J. Greenhouse Gas Control 2021. 106, 103279.
[16] a) R. F. Zheng. D. Barpaga, P. M. Mathias, D. Malhotra, P. K. Kocch, Y. Jiang, M. Bhakta, M. Lail, A. V. Rayer, G. A. Whyatt, C. J. Freeman, A. J. Zwoster, K. K. Weitz, D. J. Heldebrant, Energy Environ. Sci. 2020, 13, 4106; b) D. Malhotra, D. C. Cantu, P. K. Kocch, D. J. Heldebrant, A. Karkamkar, F. Zheng, M. D. Bearden, R. Rousseau, V.-A. Glezakou, ACS Sustainable Chem. Eng. 2019, 7, 7535; c) D. C. Cantu, D. Malhotra, M. T. Nguyen, P. K. Koech, D. Zhang, V. A. Glezakou, R. Rousseau, J. Page, R. Zheng, R. J. Perry, D. J. Heldebrant, ChemSusChem 2020, 13, 3429.
[17] S. V. Gredig, R. A. Koeppel, A. Baiker, J. Chem. Soc., Chem. Commun. 1995, https://doi.org/10.1039/c3995000007373.
[18] a) S. V. Gredig, R. Koeppel, A. Baiker, Appl. Catal., A 1997, 162, 249; b) S. V. Gredig, R. Koppel, A. Baiker, Catal. Today 1996, 29, 339; c) S. Auer, J. Mol. Catal. A: Chem. 1999, 14, 193.
[19] Y. Xic, P. Hu, T. Bendikov, D. Milstein, Catal. Sci. Technol. 2018, 8, 2784.
[20] M. K. Gnanamani, R. Garcia, G. Jacobs, K. Gora-Marek, D. C. Cronauer, A. J. Kropf, C. L. Marshall, Appl. Catal., A 2020, 602, 117722. a) A. Kaithal, B. Chatterjee, C. Werle, W. Leitner, Angew. Chem., Int. Ed. 2021, 60, 26500; b) W.-J. Shen Yasuyuki Matsumura, Phys. Chem. Chem. Phys. 2000, 2, 1519; c) Y. Liu, T. Hayakawa, T. Ishii, M. Kumagai, H. Yasuda, K. Suzuki, S. Hamakawa, K. Murata, Appl. Catal., A 2001, 210, 301; d) J. C. Brown, E. Gulari, Catal. Commun. 2004, 5, 431; c) S. A. Ranaweera, W. P. Henry, M. G. White, ACS Omega 2017, 2, 5949; f) G. Li, C. Gu, W. Zhu, X. Wang, X. Yuan, Z. Cui, H. Wang, Z. Gao, J. Cleaner Prod. 2018, 183, 415.
[22] T. Toyao, M. Nurnobi Rashed, Y. Morita, T. Kamachi, S. M. A. Hakim Siddiki, M. A. Ali, A. S. Touchy, K. Kon, Z. Maeno, K. Yoshizawa, K. i. Shimizu, ChemCatChem 2018, 11, 449.
[23] S. Bagheri, N. Muhd Julkapli, S. Bee Abd Hamid, Sci. World J. 2014, 2014, 727496.
[24] A. K. Talukdar, K. G. Bhattacharyya, S. Sivasanker, Appl. Catal., A 1993, 96, 229.
[25] J. J. Spivey, A. Egbebi, Chem. Soc. Rev. 2007, 36, 1514.
[26] a) V. Subramani, S. K. Gangwal, Energy Fuels 2008, 22, 814; b) D. Xu, Y. Q. Wang, M. Y. Ding, X. L. Hong, G. L. Liu, S. C. E. Tsang, Chem 2021, 7, 849.
[27] D. Wang, Q. Y. Bi, G. H. Yin, W. L. Zhao, F. Q. Huang, X. M. Xie, M. H. Jiang, Chem. Commun. 2016, 52, 14226.
[28] K. He, Q. Wang, Catalysts 2021, 11, 1280.
[29] a) J. Liu, F. R. Lucci, M. Yang, S. Lee, M. D. Marcinkowski, A. J. Therrien, C. T. Williams, E. C. Sykes, M. Flytzani-Stephan opoulos, J. Am. Chem. Soc. 2016, 138, 6396; b) A. I. Boronin, E. M. Slavinskaya, A. Figueroba, A. I. Stadnichenko, T. Y. Kardash, O. A. Stonkus, E. A. Fedorova, V. V. Muravev, V. A. Svetlichnyi, A. Bruix, K. M. Neyman, Appl. Catal., B 2021, 286, 119931; c) M. D. Argyle, C. H. Bartholomew, Catalysts 2015, 5, 145.
[30] L. Li, S. Miyazaki, S. Yasumura, K. W. Ting, T. Toyao, Z. Maeno, K.-i. Shimizu, ACS Catal. 2022, 12, 2639.
[31] A. Rödl, C. Wulf, M. Kaltschmitt, in Hydrogen Supply Chains, Academic Press, San Diego, CA 2018, p. 81-109.
[32] R. E. James, D. Kearins, M. Turner, M. Woods, N. Kuehn, A. Zoelle, NETL, Cost and Performance Baseline for Fossil Energy Plants, Vol. 1, Bituminous Coal and Natural Gas to Electricity 2019.
[33] Methanol production capacity may quintuple on decarbonized industry transformation: Study, https://cleanenergynews.ihsmarkit.com/research-analysis (accessed: September 2022).
[34] https://www.parrinst.com/products/stirred-reactors/series-4560-100-600-ml-mini-reactors/ (accessed: September 2022).
[35] E. D. Walter, L. Qi, A. Chamas, H. S. Mehta, J. A. Sears, S. L. Scott, D. W. Hoyt, J. Phys. Chem. C 2018, 122, 8209.

1. A method of converting CO₂ to methanol comprising:
contacting a solution comprising CO₂ in an amine sorbent over a solid catalyst comprising a noble metal on a reducible metal oxide support at a temperature in the range of 181° C. to 225° C.;
wherein the CO₂ is post or pre combustion CO₂; and
wherein the conversion of CO₂ occurs in a condensed phase and the CO₂ is converted to methanol with at least 40% selectivity; wherein the single pass conversion of CO₂ is at least 75%; and wherein the C₂₊ alcohol selectivity, or the ethanol selectivity, is at least 4 mol %.

2. The method of claim 1 further comprising one or any combination of the following: wherein the methanol selectivity is at least 50 mol %; wherein the methanol selectivity is in the range of 40 to about 80 mol %; wherein the single pass conversion of CO₂ is in the range of 75 to about 90%; wherein the selectivity to ethanol is 4 to 10 mol %; wherein the selectivity to butanol is at least 1.7 mol %; wherein the selectivity to butanol is about 2 to about 5 mol %.

3. The method of claim 1 wherein the conversion of CO₂ is in a continuous flow operation.

4. The method of claim 1 occurring in a single pass.

5. The method of claim 1 comprising a single pass CO₂ conversion of 80-90%.

6. The method of claim 1 in which the catalyst support comprises CeO₂ or TiO₂.

7. The method of claim 1 in which the amine sorbent is a water-lean solvent.

8. The method of claim 1 further comprising separation and conversion of CO₂ from landfill gases, waste-water treatment gases, manure off-gas, fermenters, cement plants, steel kilns, and pulp and paper mills.

9. The method of claim 1 in which the amine sorbent is N-(2-EthoxyEthyl)-3-MorpholinoPropan-1-Amine (2-EEMPA).

10. The method of claim 1 in the amine sorbent with 0.5-20wt % CO₂ loading.

11. The method of claim 1 in which ethanol is a co-solvent.

12. The method of claim 1 in which CO and N-formamide are formed as intermediates.

13-18. (canceled)

19. A method of converting CO2 comprising:
providing continuous flow reactor system comprising:
an absorber;
a pump; and
a reactor;
enriching an amine sorbent with CO₂ by contacting the solvent with a flue gas comprising CO₂ to create a CO₂-enriched solution in the absorber;
pressurizing the CO₂-enriched solution to between 30 and 100 bar H₂;
heating the CO₂-enriched solution;
producing a heated and pressurized CO₂-enriched solution;
feeding the heated and pressurized CO₂-enriched solution with pressurized H₂ gas into a reactor comprising a solid catalyst comprising a noble metal on a reducible metal oxide support and reacting the solution over the solid catalyst at a temperature of between 181 and 225° C.; and
producing a product solution in which the CO₂ in the solution is converted into methanol, C₂₊ alcohols, CH₄, and light hydrocarbons over the solid catalyst.

20. The method of claim 19 comprising a single pass CO₂ conversion of 80-90%.

21. The method of claim 20 in which the conversion of CO₂ occurs in a condensed phase.

22. The method of claim 19 wherein the solid catalyst comprises Pt disposed on a reducible metal oxide support.

23. The method of claim 22 wherein the catalyst support comprises CeO₂ and/or TiO₂.

24. The method of claim 19 wherein the amine sorbent is a water-lean post-combustion solvent.

25-40. (canceled)

41. A composition of matter comprising an amine solvent, CO₂, and a heterogenous catalyst, and methanol derived from the carbon dioxide, wherein the methanol derived from the CO₂ makes up at least 1% of mass percent of the CO₂ and wherein the molar ratio of the methanol produced from CO₂ to ethyl formate in the composition is greater than 2.

42. The composition of claim 41 in which the catalyst suppresses N-methylation of capture solvent.

43-45. (canceled)
